SEQUENCE LISTING (8 sequences)

SEQ ID NO: 1 - DNA, 60 bp, Artificial Sequence
  Synthetic
actcgagtataagcaaaaaatcaatcaaaacaagtaataacatggaggcccagaataccc

SEQ ID NO: 2 - DNA, 60 bp, Artificial Sequence
  Synthetic
aaaagcagaaaggcggctcaattgatagtactttgcttcagtatagcgaccagcattcac

SEQ ID NO: 3 - DNA, 59 bp, Artificial Sequence
  Synthetic
agaacgtagatagtatcatcaatcgaataataaaaaaaaacagctgaagcttcgtacgc

SEQ ID NO: 4 - DNA, 62 bp, Artificial Sequence
  Synthetic
atataaatatacctatgtttttatatgtctgtttactgatgcataggccactagtggatctg

SEQ ID NO: 5 - DNA, 19 bp, Artificial Sequence
  Synthetic
cagctgaagcttcgtacgc

SEQ ID NO: 6 - DNA, 22 bp, Artificial Sequence
  Synthetic
gcataggccactagtggatctg

SEQ ID NO: 7 - DNA, 59 bp, Artificial Sequence
  Synthetic
cgcggatccagtgcaattaagaaacgtgttacaaagaaggaagtcttcggttaacaatg

SEQ ID NO: 8 - DNA, 59 bp, Artificial Sequence
  Synthetic
cgcggatcctccagagtgaatccgggaaacataatcgggaaaattcattgttaaccgaa

(19) United States
(12) Patent Application Publication
(10) Pub. No.: US 2006/0189551 A1
(43) Pub. Date: Aug. 24, 2006

(54) Combination therapies for fungal pathogens

(75) Inventors: Joseph Heitman, Durham, NC (US); Miguel Arevalo-Rodriguez, Sevilla (ES); Xuewen Pan, Baltimore, MD (US); Jef Boeke, Baltimore, MD (US)

Correspondence Address: MEDLEN & CARROLL, LLP;Suite 350, 101 Howard Street, San Francisco, CA, 94105, US

(73) Assignees: Duke University, Durham, NC (US); Johns Hopkins University, Baltimore, MD (US)

(21) Appl. No.: 11/242,233

(22) Filed: Oct. 3, 2005

Related U.S. Application Data
(60) Provisional application No. 60/615,698, filed on Oct. 4, 2004.

(51) Int. Cl. A61K 31/7048; A61K 31/496; A61K 31/4196; A61K 31/4178

(52) U.S. Cl. 514/28; 514/254.07; 514/383; 514/397

(57) ABSTRACT

The present invention relates to methods of treating fungal infections with a drug combination: a first compound that inhibits the activity or expression of the protein FKBP12 and a second compound that inhibits the activity or expression of homoserine dehydrogenase. Evidence indicates that dual inhibitors of proteins FKBP12 and homoserine dehydrogenase are lethal to fungi. Such an approach should be minimally toxic since this combination therapy targets a biosynthetic pathway that is conserved in fungi but not in mammals.

### FIELD OF THE INVENTION

The present invention relates to methods of treating fungal infections with a drug combination comprising: a first compound that inhibits the activity or expression of the protein FKBP12 and a second compound that inhibits the activity or expression of homoserine dehydrogenase.

### BACKGROUND

The present invention relates to antifungal therapies. Transplant patients, cancer chemotherapy patients, AIDS patients and others in immunocompromised conditions are predisposed to fungal infections that can often be life-threatening. Pathogenic fungi represent an increasing clinical challenge because existing antifungal agents are hampered by issues of efficacy, toxicity and the development and/or discovery of strains that are resistant to current antifungal drugs. Since fungus and host are both eukaryotic, identifying compounds specifically toxic for the fungus but not for the host has proved to be challenging. Thus, there is a need to identify antifungal agents and therapies for use in situations where existing agents are not sufficiently effective. Additionally, there is a need for broad-spectrum antifungal agents that provide reduced toxicity to patients.

### SUMMARY OF THE INVENTION

The present invention relates to methods of treating fungal infections with combination therapies based on compounds that inhibit the activity or expression of proteins FKBP12 and homoserine dehydrogenase. Evidence indicates that a combination of inhibitors of proteins FKBP12 and homoserine dehydrogenase are lethal to fungi. While not limited to any particular mechanism, it appears that the combination is effective because the inhibitors cause an accumulation of toxic levels of the substrate of homoserine dehydrogenase, aspartate beta-semialdehyde or a derivative thereof. Toxicity should be minimal since this combination therapy targets a biosynthetic pathway that is conserved in fungi but not in mammals.

In one embodiment, the invention comprises one or more FKBP12 inhibitors in combination with one or more homoserine dehydrogenase inhibitors, which when administered to patient provide an effective antifungal therapy. In one embodiment, this invention provides methods and materials for treating or preventing infection by a pathogenic fungus in a mammalian subject, particularly a human patient, by administering to the subject one or more compounds that inhibit the activity or expression of the protein FKBP12 in combination (whether simultaneously or in separate doses) with one or more compounds that inhibit the activity or expression of homoserine dehydrogenase. In still another embodiment, the method comprises administering to the subject an effective antifungal composition comprising a homoserine dehydrogenase inhibitor and non-immunosuppressive FKBP12 inhibitor. In one embodiment, the invention provides a formulation comprising a homoserine dehydrogenase inhibitor and a FKBP12 inhibitor (e.g. formulated in a single pill or topical solution).

In some embodiments, this invention provides a method for preventing or treating a pathogenic fungus in a subject which involves administering to the subject a composition comprising a sub-immunosuppressive amount of compounds that inhibit the activity of proteins FKBP12 and homoserine dehydrogenase, e.g., administering the compounds that inhibit the activity of proteins FKBP12 and homoserine dehydrogenase in an amount and manner which provides the intended antifungal effect without substantially suppressing a patient's immune system. Preferably the compound that inhibits the activity of protein FKBP12 is non-immunosuppressive.

Important applications of this invention include, among others, the treatment or prevention of infection in a subject by a pathogenic fungus such as Aspergillus, including invasive pulmonary aspergillosis; Blastomyces, including profound or rapidly progressive infections and blastomycosis in the central nervous system; Candida, especially C. albicans, including retrograde candidiasis of the urinary tract, e.g. in patients with kidney stones, urinary tract obstruction, renal transplantation or poorly controlled diabetes mellitus; Coccidioides (C. immitis), including chronic disease which does not respond well to other chemotherapy; Cryptococcus neoformans that can affect any organ of the body but most often occurs in the central nervous system; Histoplasma capsulatum, most often asymptomatic but occasionally producing acute pneumonia or an influenza like illness and spreading to other organs and systems in the body; Mucor usually involving the lungs and invading other tissues by means of metastatic lesions including e.g. craniofacial mucormycosis and pulmonary mucormycosis; Paracoccidioides brasiliensis, characterized by primary lesions of the lungs with dissemination to many internal organs, by conspicuous ulcerative granulomas of the mucous membranes of the cheeks and nose with extensions to the skin, and by generalized lymphangitis; and Sporothrix (S. schenckii syn. Sporotrichum schenckii) that is characterized by nodules and abscesses in the superficial lymph nodes, skin, and subcutaneous tissues, that occurs especially in humans and horses, and that is usually transmitted by entry of the fungus through a skin abrasion or wound (as from the prick of a thorn).

One aspect of this invention is the treatment or prevention of infection in a subject by pathogenic fungus which is resistant to one or more other antifungal agents, especially an agent other than a FKBP12 inhibitor or homoserine dehydrogenase inhibitor, including e.g. amphotericin B or analogs or derivatives thereof (including 14(s)-hydroxyamphotericin B methyl ester, the hydrazide of amphotericin B with 1-amino-4-methylpiperazine, and other derivatives) or other polyene macrolide antibiotics, including, e.g., nystatin, candicidin, pimaricin and natamycin; flucytosine; griseofulvin; echinocandins or aureobasidins, including naturally occurring and semi-synthetic analogs; dihydrobenzo[a]napthacenequinones; nucleoside peptide antifungals including the polyoxins and nikkomycins; allylamines such as naftifine and other squalene epoxidase inhibitors; and azoles, imidazoles and triazoles such as, e.g., clotrimazole, miconazole, ketoconazole, econazole, butoconazole, oxiconazole, terconazole, itraconazole or fluconazole, those antifungal drugs currently undergoing clinical trials.

Another aspect of the invention is the treatment or prevention of infection in a subject by a pathogenic fungus in cases in which the subject is allergic to, otherwise intolerant of, or non-responsive to one or more other antifungal agents or in whom the use of other antifungal agents is otherwise contraindicated.

Another aspect of this invention is the treatment or prevention of infection by pathogenic fungus in a subject by administration of compounds that inhibit the activity of proteins FKBP12 and homoserine dehydrogenase, preferably a non-immunosuppressive FKBP12 inhibitor, in conjunction with (whether simultaneously or separately) the administration of one or more other antifungal agents, including for example, any of the previously mentioned agents or types of agents (e.g. in combination with treatment with amphotericin B, preferably in a lipid or liposome formulation; an azole or triazole such as fluconazole, for example; an aureobasidin; dihydrobenzo[a]napthacenequinone; or an echinocandin) as well as with a different compound that inhibits the activity of FKBP12 or homoserine dehydrogenase. The compounds that inhibit the activity of proteins FKBP12 and homoserine dehydrogenase may be administered before, after or at the same time the other antifungal agent is administered. In certain embodiments, the combination therapy will permit the use of reduced amounts of one or both components, relative to the amount used if used alone.

Another aspect of this invention is the treatment or prevention of infection by a pathogenic fungus in a subject by administration of compounds that inhibit the activity of proteins FKBP12 and homoserine dehydrogenase, preferably a non-immunosuppressive FKBP12 inhibitor, in conjunction with the administration to the subject of one or more immune response modifiers, including for example, agents such as GM-CSF, M-CSF, IL-3, etc., preferably in an effective amount and regimen to alleviate neutropenia and/or other deficiency in immune function. The compounds that inhibit the activity of proteins FKBP12 and homoserine dehydrogenase may be administered before, after or at the same time the immune response modifier is administered. Furthermore, the compounds that inhibit the activity of proteins FKBP12 and homoserine dehydrogenase and immune response modifier(s) may be administered in conjunction with the administration of one or more other antifungal agents, as mentioned previously.

Still another application of this invention involves administration of compounds that inhibit the activity of proteins FKBP12 and homoserine dehydrogenase to a subject for the treatment or prevention of infection by a pathogenic fungus, where the subject is immunosuppressed or immunocompromised, e.g. as the result of genetic disorder, disease such as diabetes or HIV or other infection, chemotherapy or radiation treatment for cancer or other disease, or drug or otherwise induced immunosuppression in connection with tissue or organ transplantation or the treatment of an autoimmune disorder. Where the patient is being or will be treated with an immunosuppressive agent, e.g., in connection with a tissue or organ transplantation, the compounds that inhibit the activity of proteins FKBP12 and homoserine dehydrogenase may be co-administered with the immunosuppressive agent(s) to treat or prevent a pathogenic fungal infection (or administered separately). Use of a non-immunosuppressive FKBP12 inhibitor will be preferred to avoid unduly suppressing any residual immune function in the subject.

Another aspect of this invention is the treatment or prevention of infection by a pathogenic fungus in a subject infected, or suspected of being infected, with a retrovirus such as HIV, by administration of compounds that inhibit the activity of proteins FKBP12 and homoserine dehydrogenase, preferably a non-immunosuppressive FKBP12 inhibitor, in conjunction with the administration of one or more anti-HW therapeutics (including e.g. HIV protease inhibitors, reverse transcriptase inhibitors or anti-viral agents). The compounds that inhibit the activity of proteins FKBP12 and homoserine dehydrogenase may be administered before, after or at the same time as administration of the anti-HIV agent(s).

Another aspect of this invention is the treatment or prevention of infection by a pathogenic fungus in a subject by administration of compounds that inhibit the activity of proteins FKBP12 and homoserine dehydrogenase, preferably a non-immunosuppressive antifungal FKBP12 inhibitor, in conjunction with the administration of one or more other antibiotic compounds, especially one or more antibacterial agents, preferably in an effective amount and regiment to treat or prevent bacterial infection. Again, the compounds that inhibit the activity of proteins FKBP12 and homoserine dehydrogenase may be administered before, after or at the same time as administration of the other agent(s).

It should be noted that administration of a composition comprising an antifungal amount of one or more compounds that inhibit the activity of proteins FKBP12 and one or more compounds that inhibit the activity of homoserine dehydrogenase will be particularly useful for treating or preventing a pathogenic fungal infection in a mammalian subject where the fungus is resistant to one or more other antifungal therapies, or where the use of one or more other antifungal therapies is contraindicated, e.g., as mentioned above.

Antifungal pharmaceutical compositions containing at least one compound that inhibits the activity of protein FKBP12 and at least one compound that inhibits the activity of homoserine dehydrogenase, which is preferably a non-immunosuppressive FKBP12 inhibitor, are also provided for use in practicing the embodiments of the methods of this invention. Those pharmaceutical compositions may be packaged together with an appropriate package insert containing, inter alia, directions and information relating to their antifungal use. Pharmaceutical compositions are also provided which contain one or more compounds that inhibit the activity of proteins FKBP12 and homoserine dehydrogenase together with a second antifungal agent.

In some embodiments, the invention is a method for treating a pathogenic fungal infection in a mammalian subject comprising the step of administering to the subject a composition comprising a FKBP12 inhibitor and a homoserine dehydrogenase inhibitor. In another embodiment, the FKBP12 inhibitor is non-immunosuppressive. In another embodiment, the mammalian subject is a human patient. In another embodiment, the mammalian subject is immunocompromised. In another embodiment, the subject is immunocompromised as a result of drug or radiation treatment. In another embodiment, the subject is a recipient, or is being prepared to become a recipient, of a tissue or organ transplant. In another embodiment, the FKBP12 inhibitor and the homoserine dehydrogenase inhibitor are administered in conjunction with the administration of an immunosuppressive agent. In another embodiment, the subject is immunocompromised as a result of diabetes, HrV infection, or other disease or disorder. In another embodiment, the pathogenic fungal infection is selected from the group consisting of Aspergillosis, including invasive pulmonary aspergillosis; Blastomycosis, including profound or rapidly progressive infections and blastomycosis in the central nervous system; Candidiasis, including retrograde candidiasis of the urinary tract, e.g. in patients with kidney stones, urinary tract obstruction, renal transplantation or poorly controlled diabetes mellitus; Coccidioidomycosis, including chronic disease which does not respond well to other chemotherapy; Cryptococcosis; Histopolasmosis; Mucormycosis, including e.g. craniofacial mucormycosis and pulmonary mucormycosis; Paracoccidioidomycosis; and Sporotrichosis.

In some embodiments, the invention is a method for treating a pathogenic fungal infection in a mammalian subject where the fungus is resistant to one or more other antifungal therapies, comprising the step of administering to the subject a composition comprising a FKBP12 inhibitor and a homoserine dehydrogenase inhibitor. In anther embodiment, the fungus is resistant to one or more of amphotericin B, an analog thereof or another polyene macrolide antibiotic; flucytosine; griseofulvin; or an imidazole or triazole. In another embodiment, the imidazole or triazole is clotrimazole, miconazole, ketoconazole, econazole, butoconazole, oxiconazole, terconazole, itraconazole or fluconazole. In another embodiment the composition containing the FKBP12 inhibitor and the homoserine dehydrogenase inhibitor are administered to the subject in conjunction with at least one other antifungal agent. In another embodiment, the other antifungal agent is amphotericin B, an analog thereof or another polyene macrolide antibiotic; flucytosine; griseofulvin; or an imidazole or triazoles such as, e.g., clotrimazole, miconazole, ketoconazole, econazole, butoconazole, oxiconazole, terconazole, itraconazole or fluconazole.

In some embodiments, the invention is method for treating a pathogenic fungal infection in a mammalian subject comprising the step of administering to the subject a composition comprising: a first and second compound, wherein said first compound is selected from FK506, L-683,590, L-685,818, and rapamycin; and said second compound is RI-331.

In some embodiments, the invention is a method for treating a pathogenic fungal infection in a mammalian subject comprising the step of administering to the subject a composition comprising: a first compound selected from compounds having the following formula: 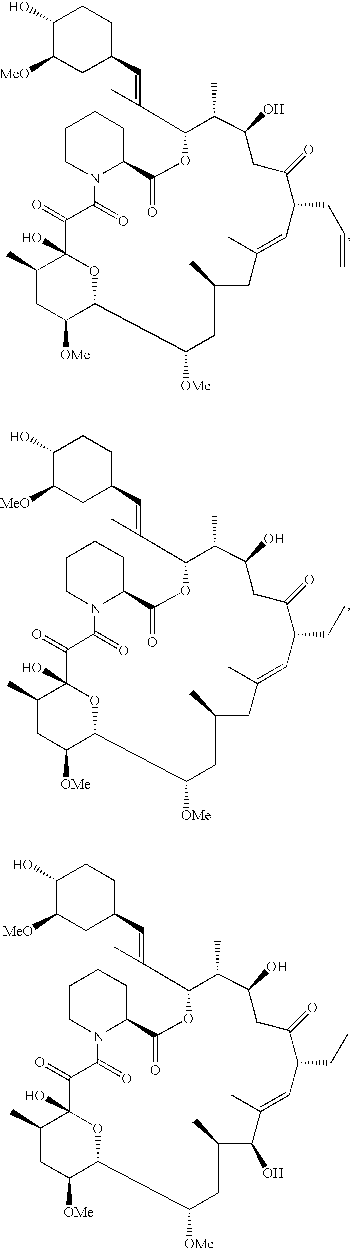 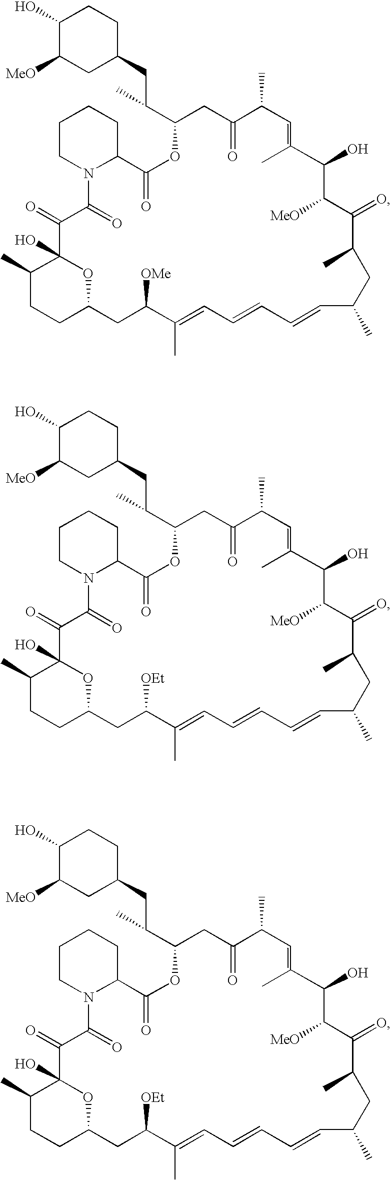 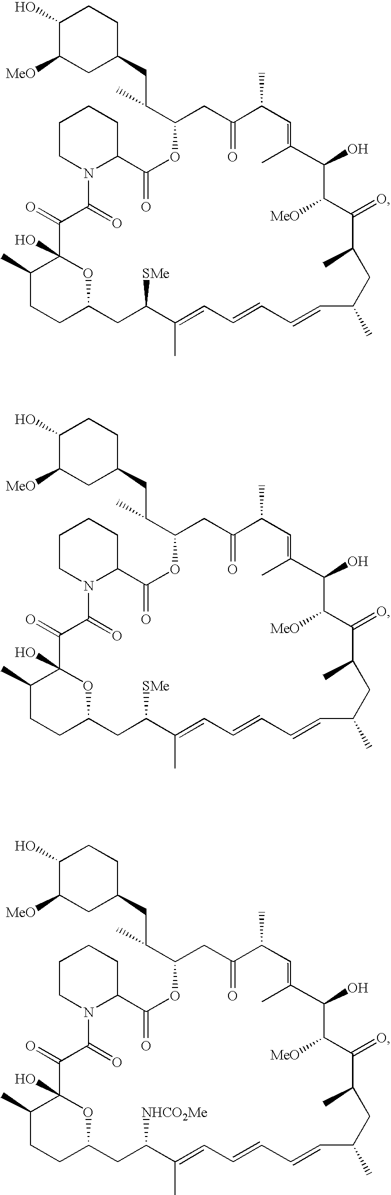 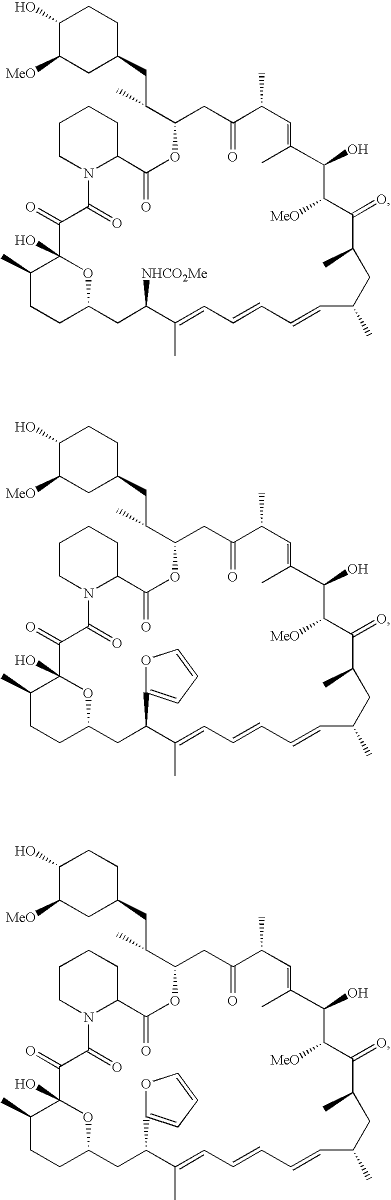 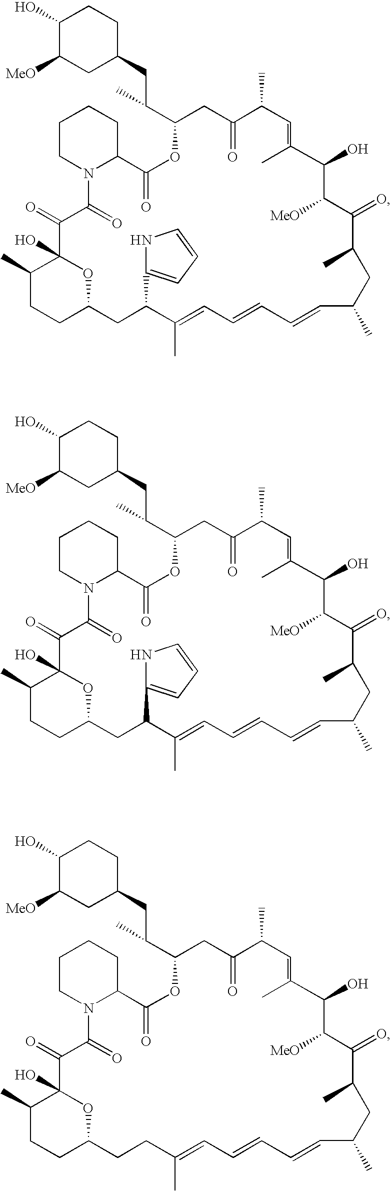 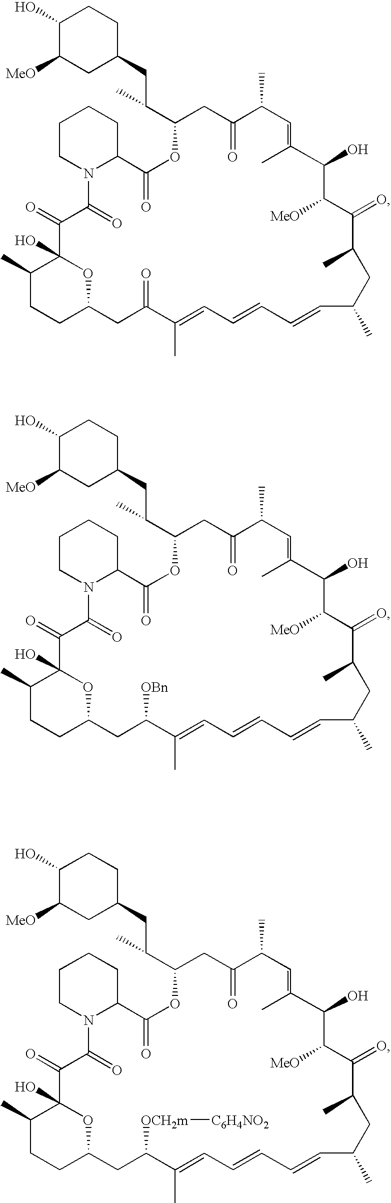 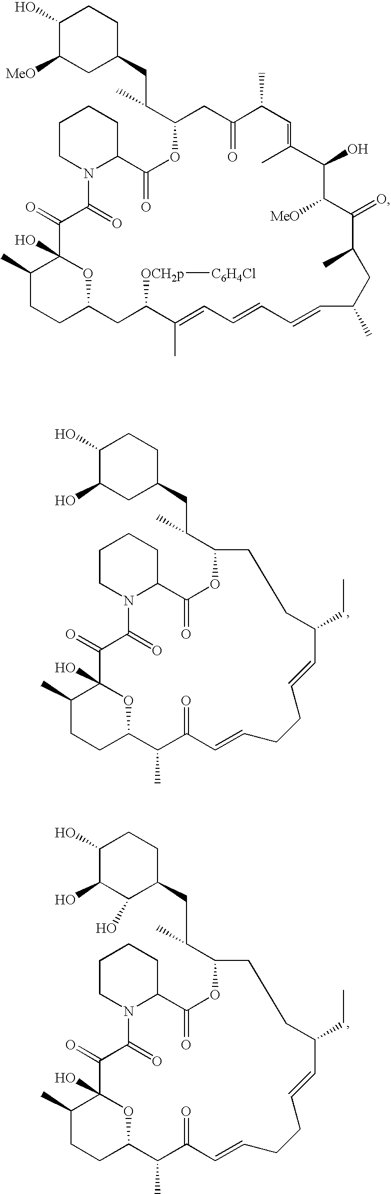 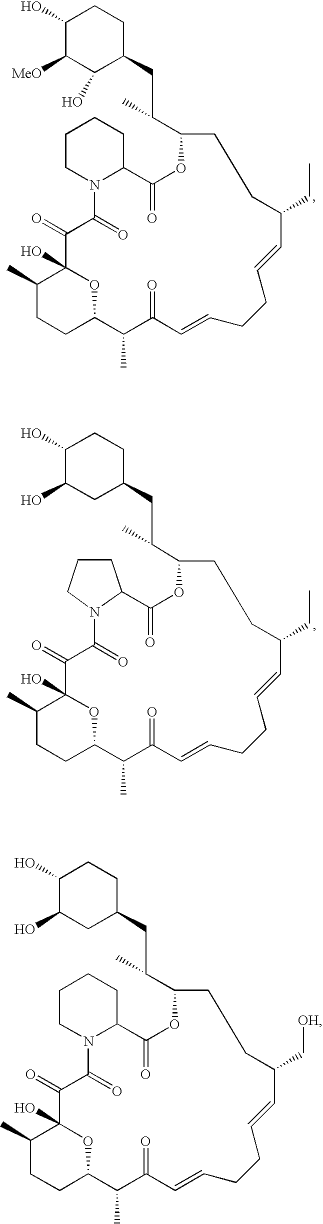 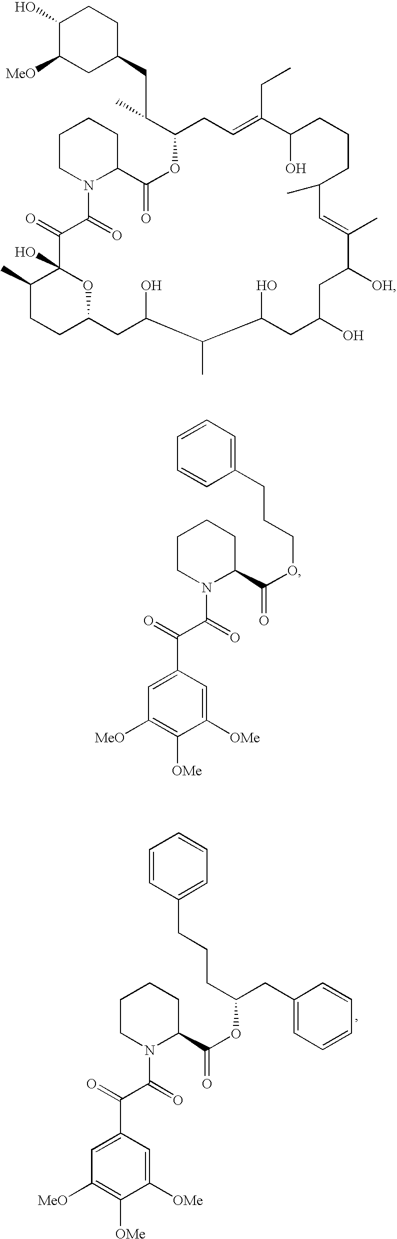 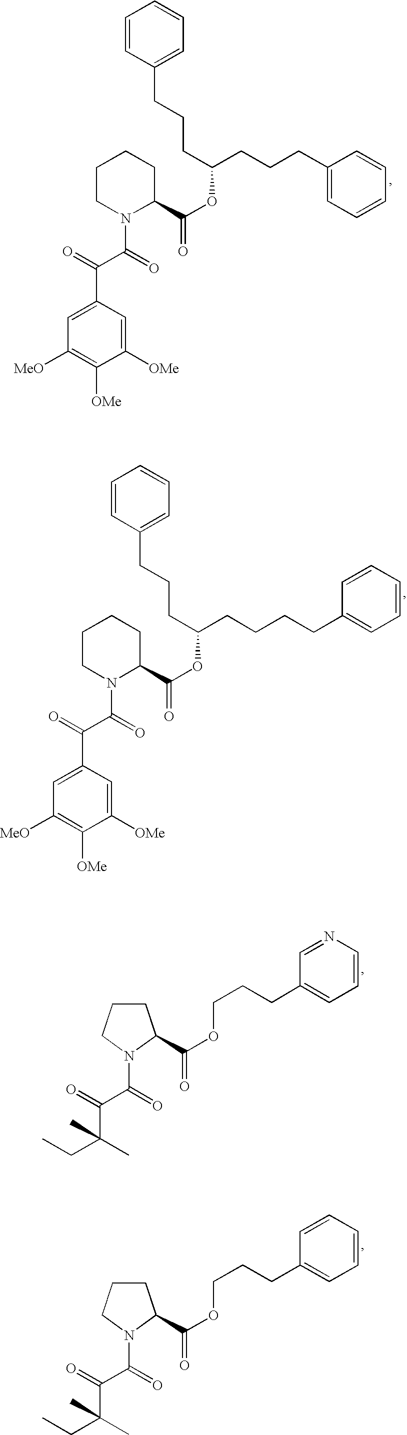 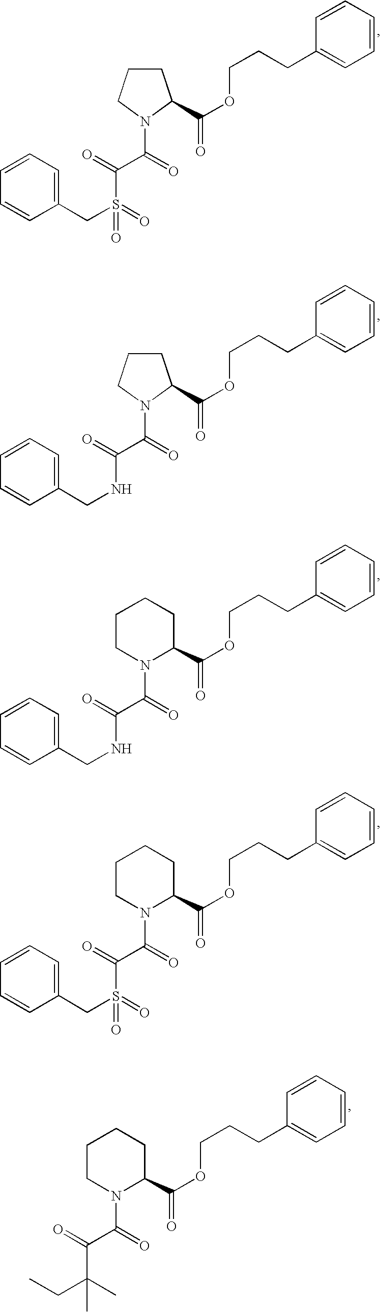 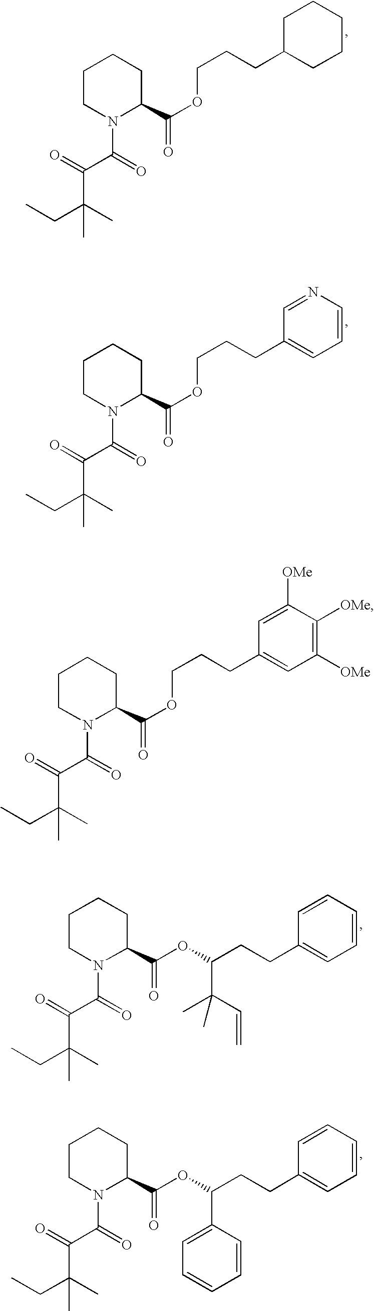 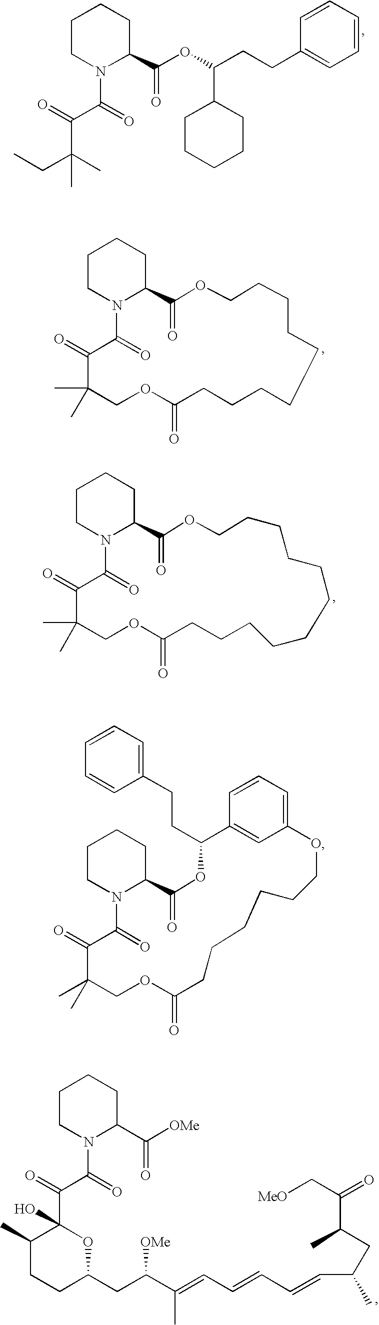 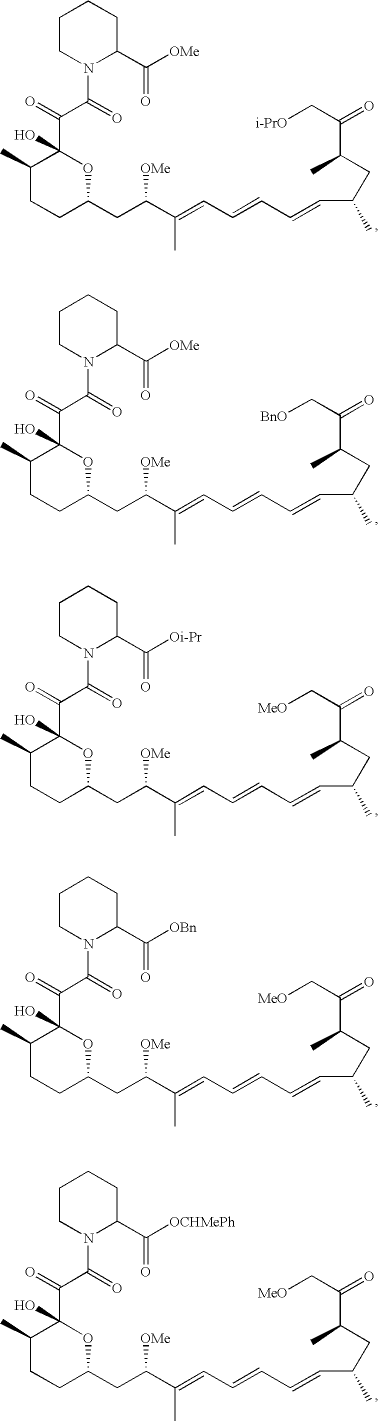 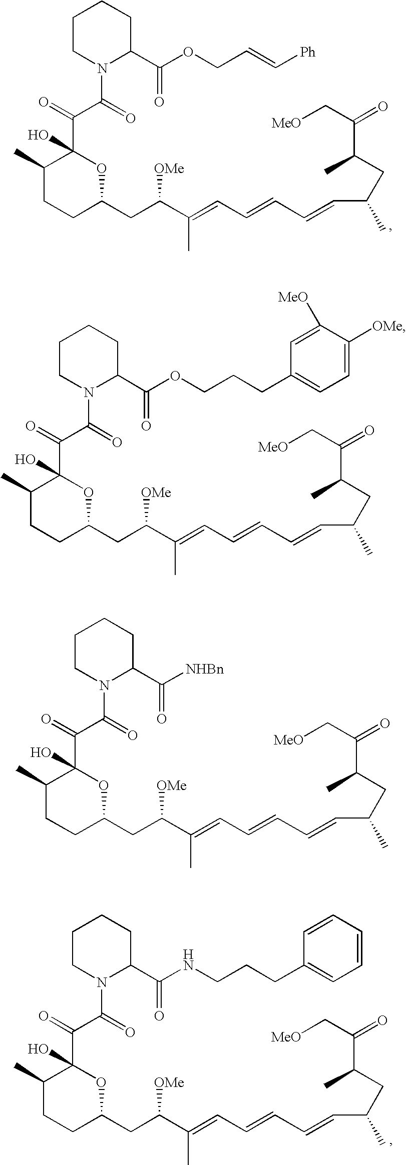 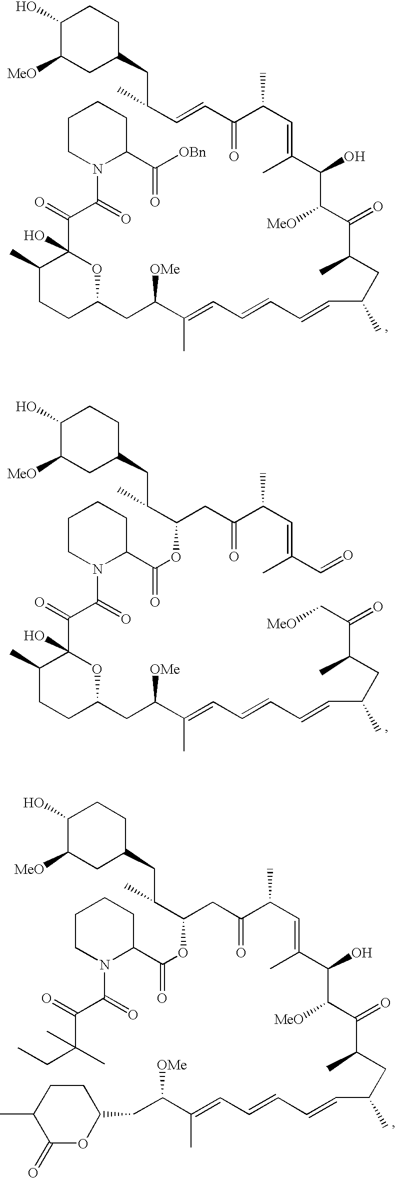 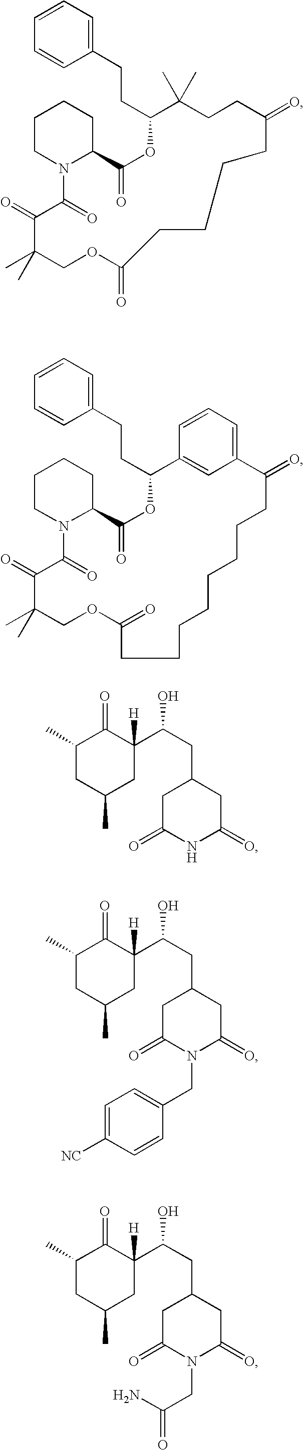 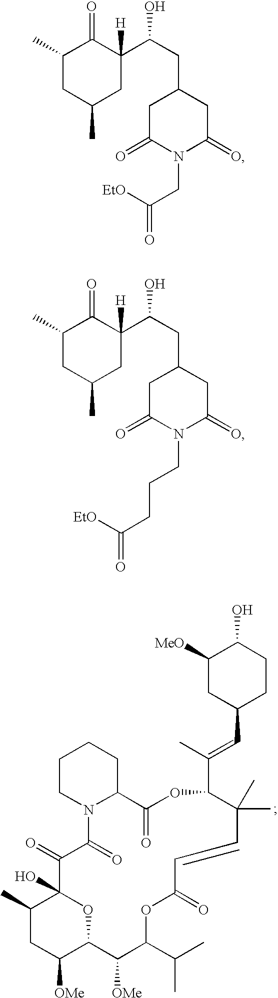
and a second compound selected from the compounds having the following formula: 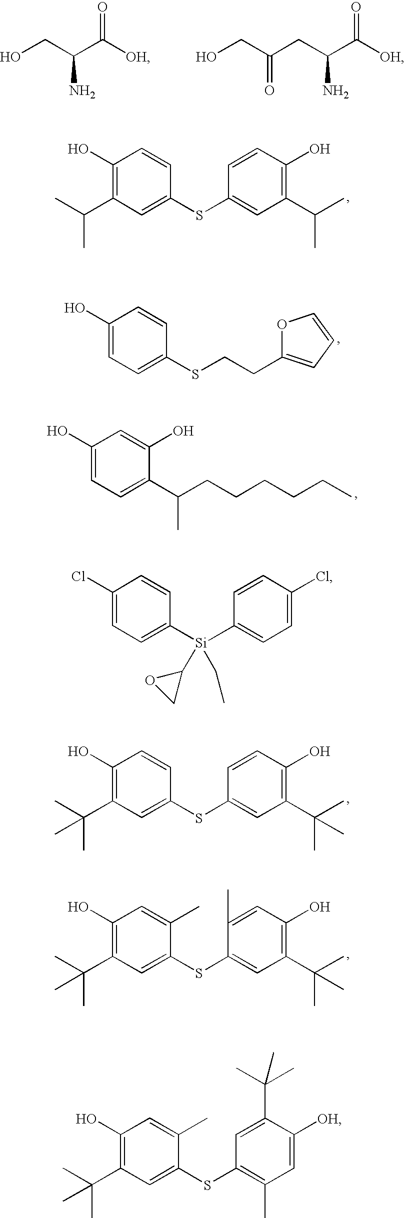 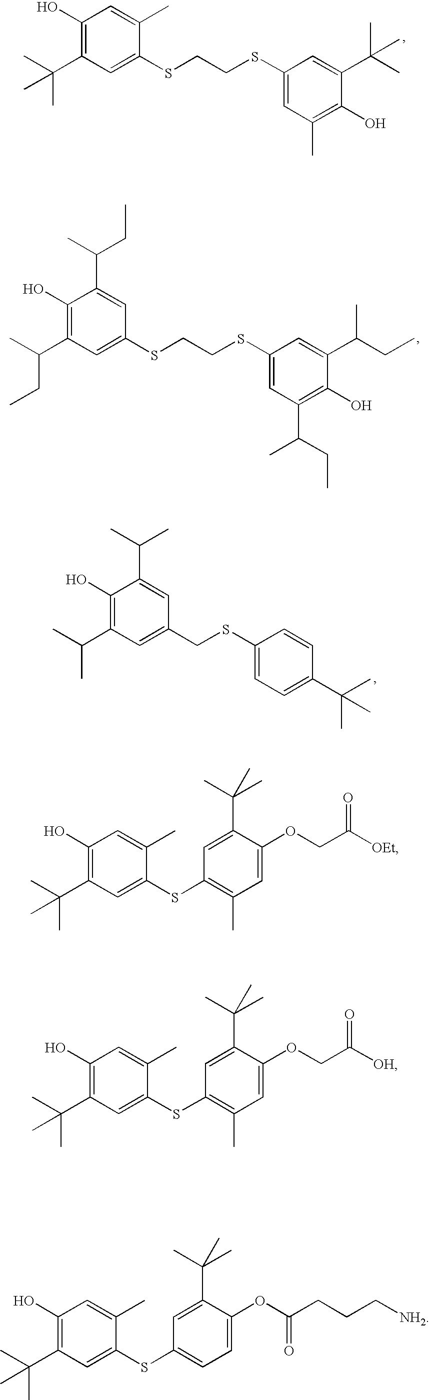
In a preferred embodiment, the mammalian subject is a human patient. In another embodiment, the subject is immunocompromised. In another embodiment, the subject is immunocompromised as a result of drug or radiation treatment. In another embodiment the subject is a recipient, or is being prepared to become a recipient, of a tissue or organ transplant. In another embodiment, the compounds are administered in conjunction (whether together or separately) with the administration of an immunosuppressive agent. In another embodiment the subject is immunocompromised as a result of diabetes, HIV infection, or other disease or disorder. In another embodiment, the pathogenic fungal infection is selected from the group consisting of Aspergillosis, including invasive pulmonary aspergillosis; Blastomycosis, including profound or rapidly progressive infections and blastomycosis in the central nervous system; Candidiasis, including retrograde candidiasis of the urinary tract, e.g. in patients with kidney stones, urinary tract obstruction, renal transplantation or poorly controlled diabetes mellitus; Coccidioidomycosis, including chronic disease which does not respond well to other chemotherapy; Cryptococcosis; Histoplasmosis; Mucormycosis, including e.g. craniofacial mucormycosis and pulmonary mucormycosis; Paracoccidioidomycosis; and Sporotrichosis. In another embodiment, the fungus is resistant to one or more other antifungal therapies. In another embodiment the fungus is resistant to one or more of amphotericin B, an analog thereof or another polyene macrolide antibiotic; flucytosine; griseofulvin; or an imidazole or triazole. In another embodiment the imidazole or triazole is clotrimazole, miconazole, ketoconazole, econazole, butoconazole, oxiconazole, terconazole, itraconazole or fluconazole. In another embodiment, the composition containing the compounds are administered to the subject in conjunction with at least one other antifungal agent. In another embodiment, the other antifungal agent is amphotericin B, an analog thereof or another polyene macrolide antibiotic; flucytosine; griseofulvin; or an imidazole or triazoles such as, e.g., clotrimazole, miconazole, ketoconazole, econazole, butoconazole, oxiconazole, terconazole, itraconazole or fluconazole.

### DESCRIPTION OF THE FIGURES

FIG. 1A. Tetrad analysis of an FPR1/fpr1Δ::nat HOM6/hom6Δ::kan diploid. Spores from strain MAYX118 were dissected on solid YPD medium, and each tetrad was arrayed in a column. Plates were incubated at 30° C. for 3 days, photographed, and replica plated to YPD plates containing 200 μg of G418/mL, 70 μg of nourseothricin (Nat)/mL, or both. Replica plates were incubated for 2 days and photographed.

FIG. 1B. Tetrad analysis of strain MAYZ118 transformed with URA3 plasmids expressing FPR1 (pYJH23) or HOM6 (pMA-HOM6) or with a URA3 control vector (pSEY8). Transformants were grown in medium selective for the plasmids (SC-uracil) and transferred to sporulation medium, and spores were dissected and analyzed as for in FIG. 1A. SC-uracil and 5-fluoro-orotic acid plates were included in this assay.

FIG. 2. FKBP12 active-site mutant restores viability of fpr1Δhom6Δ double mutants. Diploid strain MAYX118 was transformed with plasmids expressing wild-type fpr (pFP101) or the FPR1-F43Y mutant (pFP102), or with a control vector (Ycplac111), and spores were dissected and analyzed.

FIG. 3A. Aspartate pathway in yeast. A diagram of the synthesis of threonine and methionine is shown. The dotted line symbolizes feedback inhibition of the aspartokinase (AK) activity by threonine. ASD, aspartate beta-semialdehyde dehydrogenase, HD, homoserine dehydrogenase; Asp-P, beta-aspartyl phosphate; ASA, aspartate beta-semialdehyde.

FIG. 3B. fpr1Δ is not synthetically lethal with the hom3Δ and hom2Δ mutations. FprΔhom3Δ and fprΔhom2Δ double mutants are viable. Spores from FPR1/fpr1Δ::natMX4 HOM3/hom3Δ::kanMX4 (MAYX120) and FPR1/fpr1Δ::natMX4 HOM2/hom2Δ::kanMX4 (MAYX119) diploid strains were analyzed as described in FIG. 1.

FIG. 4. Interrupting the aspartate pathway suppresses lethality of fpr1Δhom6Δ double mutants. Spores from FPR1/fpr1Δ::natMX4 HOM3/hom3Δ::kanMX4 HOM6/hom6Δ::hphMX4 (MAYX122) diploid strains were dissected and the resulting colonies were analyzed by replica plating them to YPD medium containing G418, nourseothricin, 100 μg of hygromycin B (Hyg)/mL, or all three drugs combined at the same concentrations.

FIG. 5. Deletion of HOM6 is deleterious to strains expressing a mutant aspartokinase resistant to feedback inhibition. Spores from the HOM3-R7/hom3Δ::kanMX4 HOM6/hom6Δ::hphMX4 diploid strain (MAYX125) were analyzed.

FIG. 6A. Aspartokinase interacts with itself and FKBP12. FKBP12-aspartokinase (AK) and AK-AK interaction by in vitro affinity chromatography. Crude protein extracts obtained from a wild-type strain (BY4741) or from a strain expressing the HOM3-R7 mutant allele (MAY315) were incubated with His₆-tagged yeast FKPB12 coupled to agarose beads in the presence or absence of 30 mM L-threonine. Bound proteins were eluted and analyzed by Western blotting with antibodies against yeast aspartokinase. Binding reactions wit agarose beads alone were included as controls.

FIG. 6B. Fpr1-AK and AK-AK interactions in vivo. Transformants from yeast two-hybrid host strain PJ69-4A coexpressing Ga14 DNA-binding domain (BD) fusion proteins from plasmid pGBT9-Fpr1 (BD-Fpr1), pGBT9-AK (BD-AK), or pGBT9-AK(E282D) (BD-AD^{E282D}) and Ga14 activation domain (AD) fusion proteins from plasmids pGAD424-AK (AD-AK) or pGAD424-AK(E282D) (AD-AK^{E212D}) were grown in SD medium supplemented with uracil, adenine, methionine, and histidine (YNB) or in the same medium plus 1 g of L-threonine/liter (YNB+Thr) or 10 μg of FK506/mL (YNB+FK506), and induction of the lacZ reporter gene was quantified by assaying beta-galactosidase activity. PJ69-4A cotransformed with vectors pGBT9 (BD) and pGAD424 (AD) was assayed as a control.

FIG. 7A. hom6Δ mutation confers sensitivity to FK506. Approximately 10⁷ cells from hom6Δ mutant strain MAY308 were plated on solid YPD medium and exposed to 250 μg of FK506 diffusing form a paper disk. A control paper disk containing only the FK506 solvent (90% EtOH plus 10% Tween 20) was included. The plate was incubated at 30° C. for 2 days and photographed. As control, the same assay was conducted with isogenic wild-type strain BY4742.

FIG. 7B. Liquid YPD cultures of hom6 (MAY308) and wild-type (BY4742) strains were exposed to 10 μg of FK506/mL or left untreated as controls, and growth was measured based on optical density at 600 nm (OD_{600 nm}).

FIG. 7C. hom6Δ cells from the FK506-exposed culture described for FIG. 7B were photographed using differential interference contrast microscopy after 22 hours of incubation with drug.

FIG. 8A. Aspartokinase (AK) inhibition by threonine. Western blot analysis of AK and FKBP12 in protein extracts and partially purified AK preparations obtained from the wild type (BY4741) or from an fpr1Δ (MAY193), hom6Δ (MAY308, or HOM3-R7 (MAY315) mutant strain or from an hom3Δ mutant strain (MAY310) as a control.

FIG. 8B. Aspartokinase (AK) activity in partially purified extracts obtained from the strains described in FIG. 8A was assayed in the presence or absence of threonine (1 or 5 mM).

FIG. 9. Oligonucleotide primers used to produce gene specific deletions by PCR, and to diagnose the presence of specific gene deletions in engineered strains.

FIG. 10. Genotypes of the S. cerevisiae yeast strains employed in the studies to define a synergistic lethal interaction between the genes encoding FKBP12 and homoserine dehydrogenase and elucidate the mechanistic basis of this genetic interaction that can be targeted by novel drug combinations.

### DEFINITIONS

“Antifungal” means destroying or inhibiting the growth of fungi. For the various embodiments described above, it is not intended that the present invention be limited to complete inhibition of fungal growth or complete killing of fungi. It is sufficient on the one hand that growth is 50% inhibited, more preferably 80% inhibited, and most preferably 90% inhibited or more. On the other hand, it is sufficient if 50% are killed, more preferably 80% are killed, and most preferably 90% or more are killed.

“FKBP12 inhibitor”, “an inhibitor of FKBP12”, and the like, mean a chemical substance that that has an affinity for or interferes with any physiological action of a fungal FKBP12 protein. In one embodiment, such an inhibitor binds FKBP12.

“Homoserine dehydrogenase inhibitor”, “an inhibitor of homoserine dehydrogenase”, and the like, mean a chemical substance that has an affinity for or interferes with any physiological action of a fungal homoserine dehydrogenase. In one embodiment, such an inhibitor inhibits the activity of the dehydrogenase.

“Immunosuppression” means lowering the body's normal immune response to invasion by foreign substances; this can be deliberate (as in lowering the immune response to prevent rejection of a transplanted organ) or incidental (as a side effect of radiotherapy or chemotherapy for cancer, but not limited to).

“Immunocompromised” means having the immune system impaired or weakened (as by drugs, illness, genetic phenotype, and the like). The term encompasses both partially immunocompromised and completely immunocompromised.

The phrase “pharmaceutically acceptable derivative”, and the like denotes any pharmaceutically acceptable salt, ester, or salt of such ester, of such compound, or any other adduct or derivative which, upon administration to a patient, is capable of providing (directly or indirectly) compounds that inhibit the activity of FKBP12 or homoserine dehydrogenase as described herein, or a metabolite or residue thereof, and preferably, the FKBP12 inhibitor is a non-immunosuppressive agent. Pharmaceutically acceptable derivatives thus include among others pro-drugs of compounds that inhibit the activity of proteins FKBP12 and homoserine dehydrogenase. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest.

The relative antifungal activity of a given therapeutic combination, in relation to another antifungals such as rapamycin, may be quantified on a molar basis using any scientifically valid antifungal assay and a pathogenic fungal strain of interest. A variety of such antifungal assays, both in vitro and in vivo, are known in the art. By way of example, if the EC50 of a combination therapy against a given pathogenic fungal strain is 6 nM and the EC50 of rapamycin against that strain is 2 nM, the relative antifungal activity (“AF”) of the combination therapy is 0.33.

“Therapeutic immunosuppression”, as that term is used herein, denotes the level or degree of immunosuppression which characteristically results from the administration of an known immune suppressant drug, such as rapamycin, to human patients in doses appropriate for immunosuppressive purposes. Therapeutic immunosuppression thus comprises a substantial decrease in one or more aspects of the patient's immune functions including circulating T and/or B cell levels, total white blood cell counts, responsiveness of T cells to mitogenic stimulation, delayed type hypersensitivity response, etc. One appropriate in vitro surrogate of immunosuppression in a human patient is inhibition of human T cell proliferation in vitro. This is a conventional assay approach that may be conducted in a number of well known variations using various human T cells or cells lines, including among others human PBLs and Jurkat cells. A combination therapy may thus be assayed for human immunosuppressive activity and compared with a known antifungal such as rapamycin. A decrease in immunosuppressive activity relative to rapamycin measured in an appropriate in vitro assay is predictive of a decrease in immunosuppressive activity in humans, relative to rapamycin. Such in vitro assays may be used to evaluate the compound's relative immunosuppressive activity (“IS”), For example, if the EC50 of the compounds in a human T cell proliferation assay is 400 mM, while that of rapamycin is 1 nM, then the IS value for the compound is 0.0025. In the foregoing illustration, the compound had a relative antifungal activity-to-relative immunosuppressive activity (“AF/IS”) ratio of 132 (i.e., 0.33/0.0025)

The term “non-immunosuppressive” therapy or combination therapy, or the like, is used herein to denote a combination therapy that possesses an AF/IS value, with respect to at least one pathogenic fungal strain, of greater than 25, preferably greater than 100, more preferably greater than 500 and even more preferably greater than 1000. Especially preferred non-immunosuppressive combination therapies have an AF/IS ratio of 5000 or greater. Non-immunosuppressive, FKBP12 inhibitors include FKBP12 inhibitors that do not impart a therapeutic immunosuppressive effect when administered in an effective antifungal amount and dosing regimen. However, the FKBP12 inhibitor need not be completely devoid of immunosuppressive effects, but should have less than 0.1, preferably less than 0.01, and even more preferably, less than 0.005 times the immunosuppressive effect observed or expected with an equimolar amount of rapamycin, as measured clinically or in an appropriate in vitro or in vivo surrogate of human immunosuppressive activity, preferably carried out on tissues of lymphoid origin. Preferably, the selected non-immunosuppressive combination therapy does not have other toxicity to the subject when administered in a manner and amount that allows it to provide its intended antifungal effect.

The term “sub-immunosuppressive amount” of a combination therapy denotes a dose of a given FKBP12 inhibitor that is insufficient to cause theraputic immunosuppression.

The language “pharmaceutically acceptable carrier” is intended to include carriers or excipients which allow for administration of the compounds to a subject in a manner in which it performs its intended antifungal function. Carriers include e.g. pharmaceutically acceptable grades of saline, buffered saline, dextrose, water, glycerol, ethanol, and combinations thereof, and are discussed in greater detail below. The composition, if desired, can also contain minor amounts of wetting or emulsifying agents, or pH buffering agents

### DETAILED DESCRIPTION OF THE INVENTION

The present invention relates to methods of treating fungal infections such as vaginitis with combination therapies based on compounds that inhibit the activity or expression of proteins FKBP12 and homoserine dehydrogenase. While not limited to any particular mechanism, the evidence indicates that a combination of inhibitors of proteins FKBP12 and homoserine dehydrogenase are lethal to fungi because the inhibitors cause an accumulation of toxic levels of the substrate of homoserine dehydrogenase, aspartate beta-semialdehyde or a derivative thereof. This combination therapy targets a biosynthetic pathway that is conserved in fungi but not in mammals.

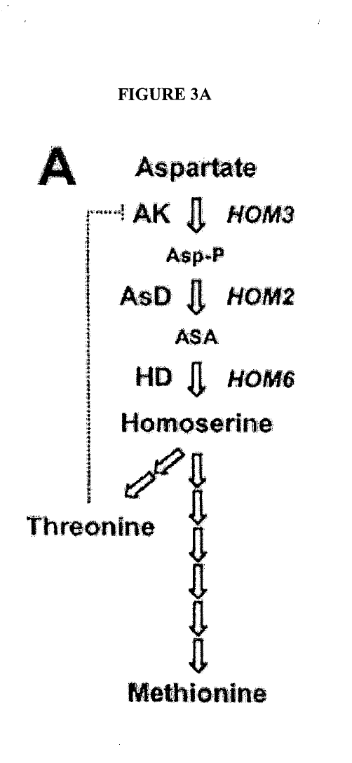

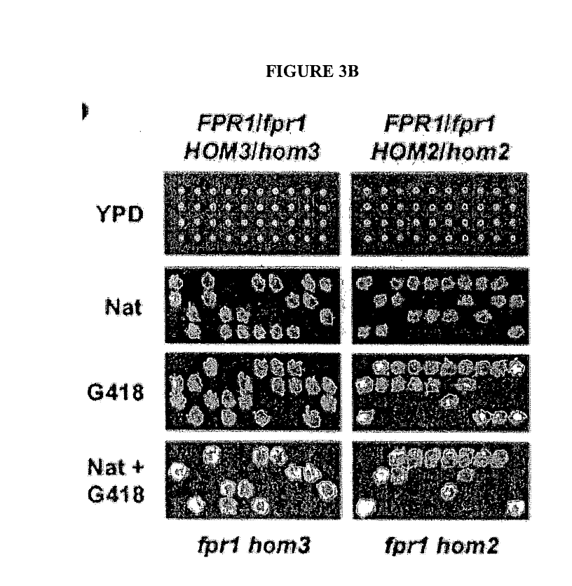

Yeast is fungus of the genus Saccharomyces. Homoserine is a precursor in biosynthesis of the amino acids threonine and methionine. The yeast HOM6 gene encodes homoserine dehydrogenase, which catalyzes the last of three steps in the conversion of aspartic acid to homoserine. (FIG. 3A) The first two steps are catalyzed by aspartokinase and aspartate beta-semialdehyde dehydrogenase, which are encoded by the HOM3 and HOM2 genes, respectively. hom3, hom2 and hom6 mutants are all auxotrophic for threonine and methionine and must therefore import these amino acids from a culture medium to survive.

Prolyl isomerases are widely conserved, ubiquitous enzymes that catalyze cis-trans isomerization of peptidyl-prolyl bonds, a reaction that can be rate limiting for protein folding. FKBP12, otherwise known as FK506 (tacrolimus) binding protein, is a prolyl isomerase. Human FKBP12 normally binds FK506 forming a complex that inhibits the functions of calcineurin in T-cell activation. It is believed to be one of the mechanisms by which FKBP12 can be manipulated to provide mammalian immune suppression. The FKBP12 protein is substantially conserved in budding yeast S. cerevisiae where it is encoded by the gene FPR1.

The FPR1 and HOM6 genes, encode proteins FKBP12 and homoserine dehydrogenase respectively. The inventors discovered that individual FPR1 mutants did not show growth delay when incubated in rich YEPD medium when compared to wild type. This was also true for individual HOM6 mutants. However, FPR1 and HOM6 (fpr1Δhom6Δ) double mutants did not thrive in rich YEPD medium.

Although the inventors do not intend the invention to be limited to any particular mechanism, the inventors believe that inhibitors of proteins FKBP12 and homoserine dehydrogenase are lethal to fungi because of the accumulation of toxic levels of aspartate beta-semialdehyde or derivative thereof. FKBP12 interacts with aspartokinase downregulating aspartokinase's ability to convert aspartic acid into beta-aspartyl phosphate. FKBP12 inhibitors prevent aspartokinase from interacting with FKBP12. Therefore, inhibiting FKBP12-aspartokinase interactions increases the enzymatic production of aspartate beta-semialdehyde. Because homoserine dehydrogenase does not convert aspartate beta-semialdehyde to homoserine in the presence of a homoserine dehydrogenase inhibitor, the biosynthetic conversion of aspartic acid by aspartokinase ultimately results in a toxic accumulation of aspartate beta-semialdehyde preventing fungal growth.

This proposed mechanism is evidenced by additional experiments showing that the expression of an FKBP12 mutant protein with reduced prolyl-isomerase activity restores viability of fpr1Δhom6Δ double mutants. fpr1 is not synthetically lethal with the other HOM mutations, and deletion of HOM3 or HOM2 suppresses lethality of fpr1Δhom6Δ double mutants. Deletion of HOM6 is deleterious to strains expressing an aspartokinase resistant to feedback inhibition. FKBP12-aspartokinase interactions are induced by threonine, and reduced by a mutation in HOM3 that renders aspartokinase resistant to feedback inhibition. The homΔ6 mutation confers sensitivity to FK506, and aspartokinase from an fpr1Δ mutant is inhibited by threonine in vitro.

It is not intended that the present invention be limited to particular inhibitors. The invention contemplates numerous different FKBP12 inhibitors and homoserine dehydrogenase inhibitors, which when administered to a patient in combination are effective antifungal therapies because the combination therapy targets a biosynthetic pathway that is conserved in fungi but not in mammals. Additionally, the applicant has identified combination therapies that are non-immunosuppressive in mammals.

Fungi that are pathogenic in humans that can be prevented or treated by the currently proposed combination therapies include, among others, Candida species including C. albicans, C. tropicalis, C. kerr (?), C. krusei and C. galbrata; Aspergillus species including A. fumigatus and A. flavus; Cryptococcus neoformans; Blastomyces species including Blastomyces dermatitidis; Pneumocystis carinii; Coccidioides immitis; Basidiobolus ranarum; Conidiobolus species; Histoplasma capsulatum; Rhizopus species including R. oryzae and R. microsporus; Cunninghamella species; Rhizomucor species; Paracoccidioides brasiliensis; Pseudallescheria boydii; Rhinosporidium seeberi; and Sporothrix schenckii.

FKBP12 Inhibitors

Rapamycin and structurally similar tacrolimus (FK506) and derivatives thereof are inhibitors of FKBP12. Prior to becoming a well-known immune suppressant, Rapamycin was first described as an antifungal agent. For example U.S. Pat. No. 6,258,823, hereby incorporated by reference, discloses derivatives of rapamycin, their ability to inhibit FKBP12, and their antifungal uses.

A large number of structural variants of rapamycin and tacrolimus (FK506) that are inhibitors of FKBP12 have been reported, typically arising as alternative fermentation products or from synthetic efforts to improve the compound's therapeutic index as an immunosuppressive agent. For example, the extensive literature on analogs, homologs, derivatives and other compounds related structurally to rapamycin include among others variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

The following disclose how to make such variants and others, and are hereby incorporated by reference: WO 7/10502, WO 94/18207, WO 93/04680, U.S. Pat. No. 5,527,907, U.S. Pat. No. 5,225,403, WO 96/41807, WO 94/10843, WO 92/14737, U.S. Pat. No. 5,484,799, U.S. Pat. No. 5,221,625, WO 96/35423, WO 94/09010, WO 92/05179, U.S. Pat. No. 5,457,194, U.S. Pat. No. 5,210,030, WO 96/03430, WO 94/04540, U.S. Pat. No. 5,604,234, U.S. Pat. No. 5,457,182, U.S. Pat. No. 5,208,241, WO 96/00282, WO 94/02485, U.S. Pat. No. 5,597,715, U.S. Pat. No. 5,362,735, U.S. Pat. No. 5,200,411, WO 95/16691, WO 94/02137, U.S. Pat. No. 5,583,139, U.S. Pat. No. 5,324,644, U.S. Pat. No. 5,198,421, WO 95/15328, WO 94/02136, U.S. Pat. No. 5,563,172, U.S. Pat. No. 5,318,895, U.S. Pat. No. 5,147,877, WO 95/07468, WO 93/25533, U.S. Pat. No. 5,561,228, U.S. Pat. No. 5,310,903, U.S. Pat. No. 5,140,018, WO 95/04738, WO 93/18043, U.S. Pat. No. 5,561,137, U.S. Pat. No. 5,310,901, U.S. Pat. No. 5,116,756, WO 95/04060, WO 93/13663, U.S. Pat. No. 5,541,193, U.S. Pat. No. 5,258,389, U.S. Pat. No. 5,109,112, WO 94/25022, WO 93/11130, U.S. Pat. No. 5,541,189, U.S. Pat. No. 5,252,732, U.S. Pat. No. 5,093,338, WO 94/21644, WO 93/10122, U.S. Pat. No. 5,534,632, U.S. Pat. No. 5,247,076, U.S. Pat. No. 5,091,389 and U.S. Pat. No. 5,162,333.

The following references disclose compounds having an affinity for the FK-506 binding protein (FKBP12), and are hereby incorporated by reference: U.S. Pat. No. 5,622,970, U.S. Pat. No. 5,516,797, U.S. Pat. No. 5,330,993, U.S. Pat. No. 5,192,773, U.S. Pat. No. 5,801,197, U.S. Pat. No. 5,801,187, U.S. Pat. No. 5,798,355, U.S. Pat. No. 5,795,908, U.S. Pat. No. 5,786,378,

The following references disclose compounds that are neurotrophic agents (i.e. compounds capable of stimulating growth or proliferation of nervous tissue) and that bind to immunophilins such as FKBP12 and inhibit their peptidyl-prolyl isomerase or rotamase activity. The following references, hereby incorporated by reference, describe such compounds: U.S. Pat. No. 6,239,146, U.S. Pat. No. 6,228,872, U.S. Pat. No. 6,096,762, U.S. Pat. No. 5,721,256, U.S. Pat. No. 5,696,135, and U.S. Pat. No. 5,780,484.

U.S. Pat. No. 5,935,954 discloses compounds and pharmaceutical compositions for treatment of multi-drug resistant cells that have the ability to bind the cellular protein FKBP12, and is hereby incorporated by reference.

U.S. Pat. No. 6,133,456 discloses compounds for multimerizing immunophilins and proteins containing immunophilin or immunophilin-related domains such as FKBP12 and is hereby incorporated by reference.

FK506, [3S-[3R*[E(1S*,3S*,4S*)],4S*,5R*,8S*,9E,12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone is a FKBP12 inhibitor of the following formula:

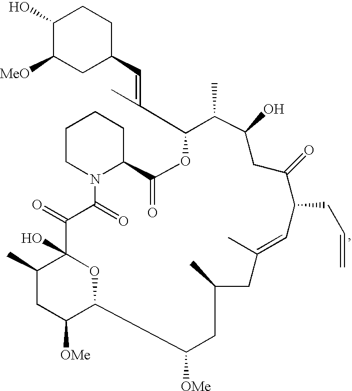

L-683,590 is a FKBP12 inhibitor of the following formula:

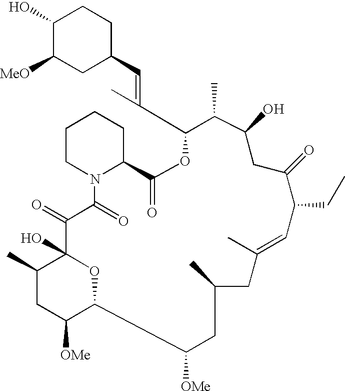

L-685,818 is a FKBP12 inhibitor of the following formula:

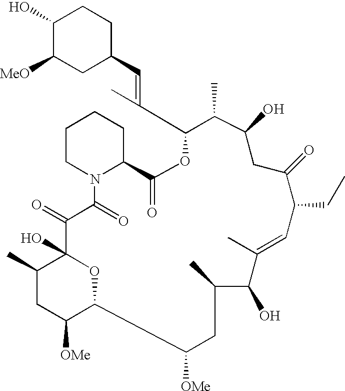

Rapamycin, (3S,6R,7E,9R,10R,12R,14S,15E,17E,19E,21 S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1c][1,4]oxaazacyclohentriacontine-1,5,11,28,29 (4H,6H,31H)-pentone is a FKBP12 inhibitor of the following formula:

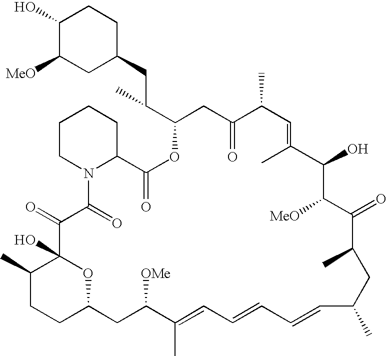

There are numerous known FKBP12 inhibitors including, but not limited to, those disclosed in Luengo et al., Chemistry & Biology 1995 7(2), 471-481, hereby incorporated by reference, Fehr et al., J. Antibiotics 1996 49(3) 230-233, hereby incorporated by reference, Salituro et al., Tetrahedron Letters 1995 36(7) 977-1000, hereby incorporated by reference, Christner et al., J. Med. Chem. 1999, 42, 3615-3622, hereby incorporated by reference, Armistead et al., Acta Cryst. (1995). DSI, 522-528, hereby incorporated by reference, Wei et al., Bioorg. Med. Chem. Lett. 12 (2002) 1429-1433, hereby incorporated by reference, Holt et al., J. Am. Chem. Soc. 1993, 115, 9925-9938, hereby incorporated by reference, Holt et al., Bioorganic & Medicinal Chemistry Letters. Vol. 3, No. 10. pp. 1977-1980.1593, hereby incorporated by reference, Yamashita et al., Bioorganic & Medicinal Chemistry Letters Vol. 4. NO. 2, pp. 325-328, 1994, hereby incorporated by reference, Bierer et al., Science. 1990 250(4980) 556-9, hereby incorporated by reference, and preferably those of the following formula: 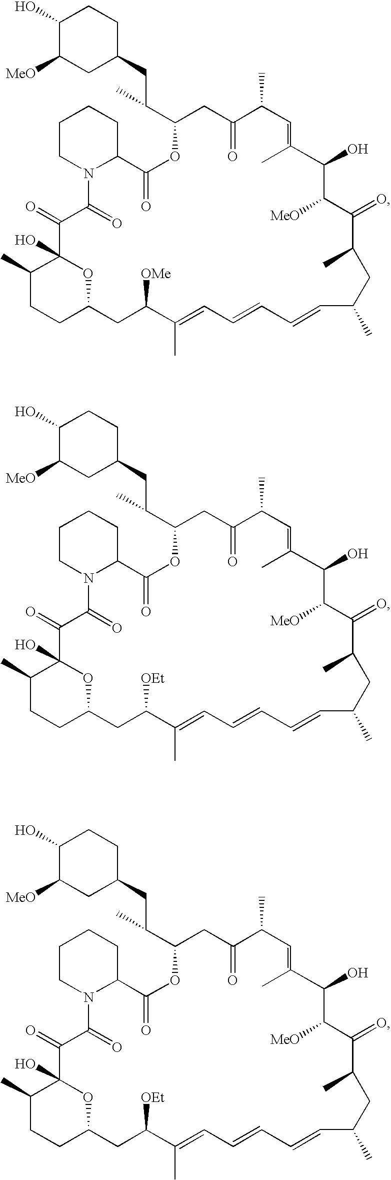 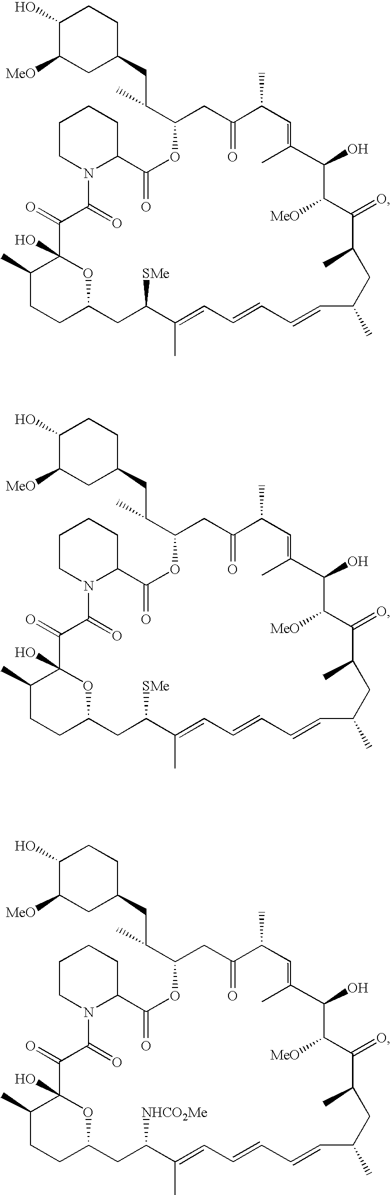 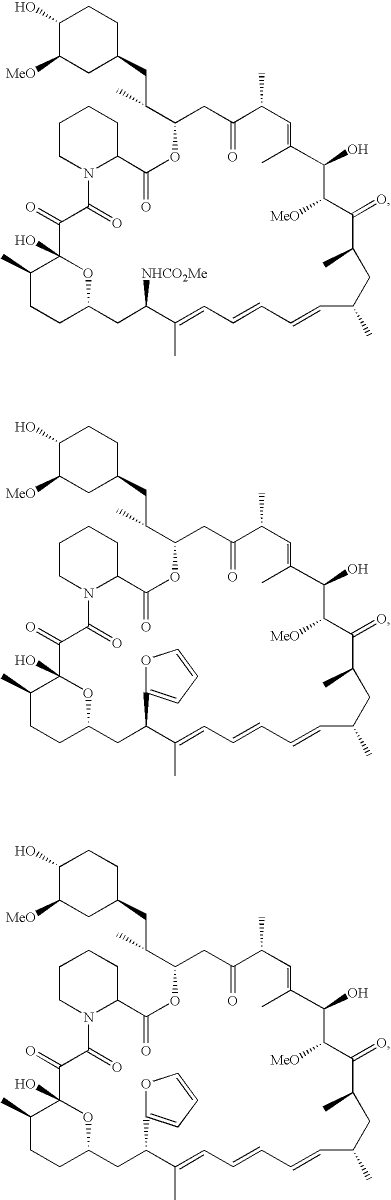 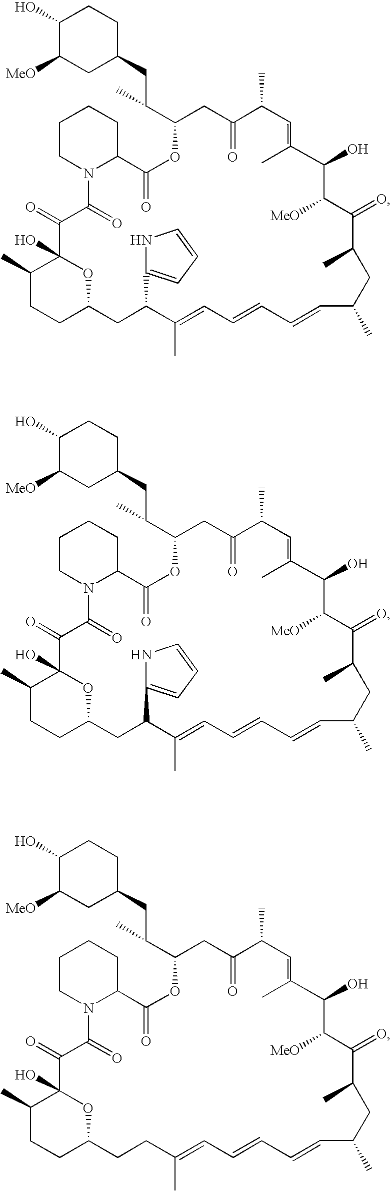 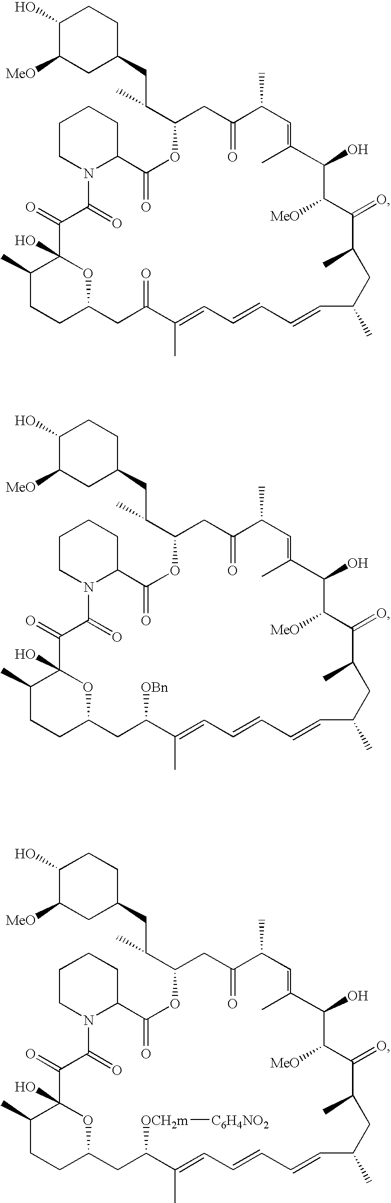 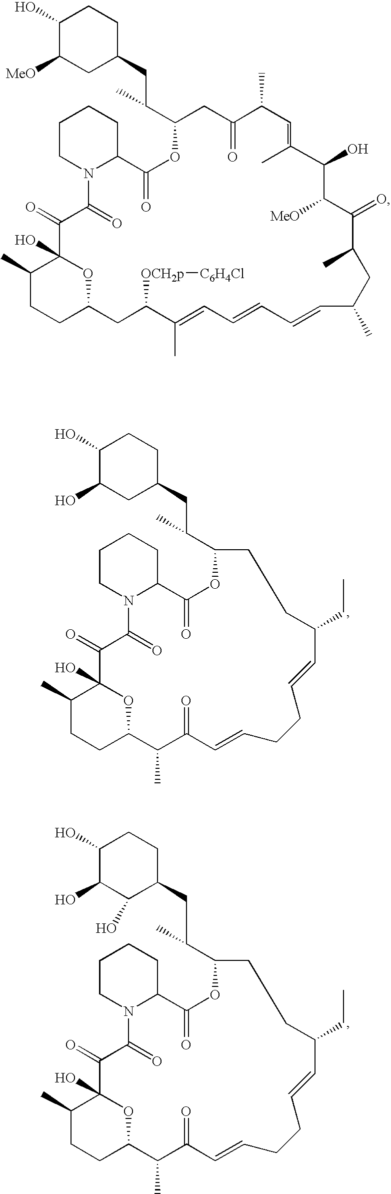 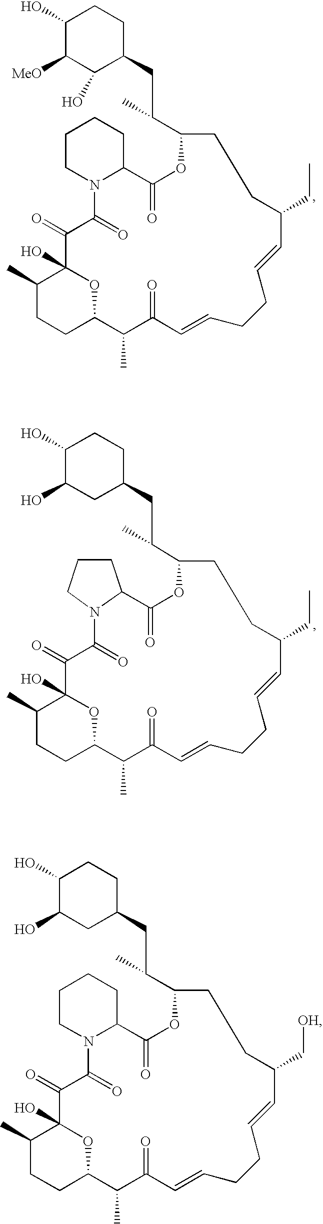 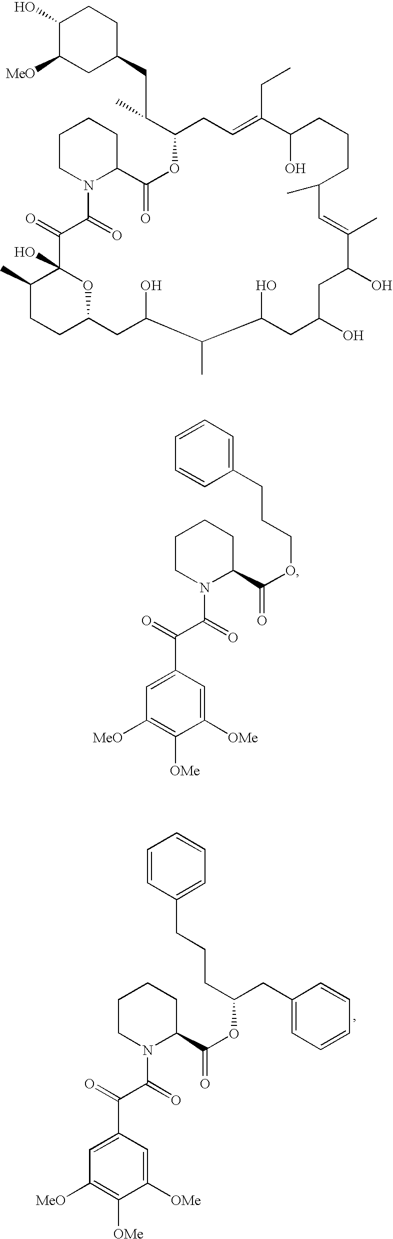 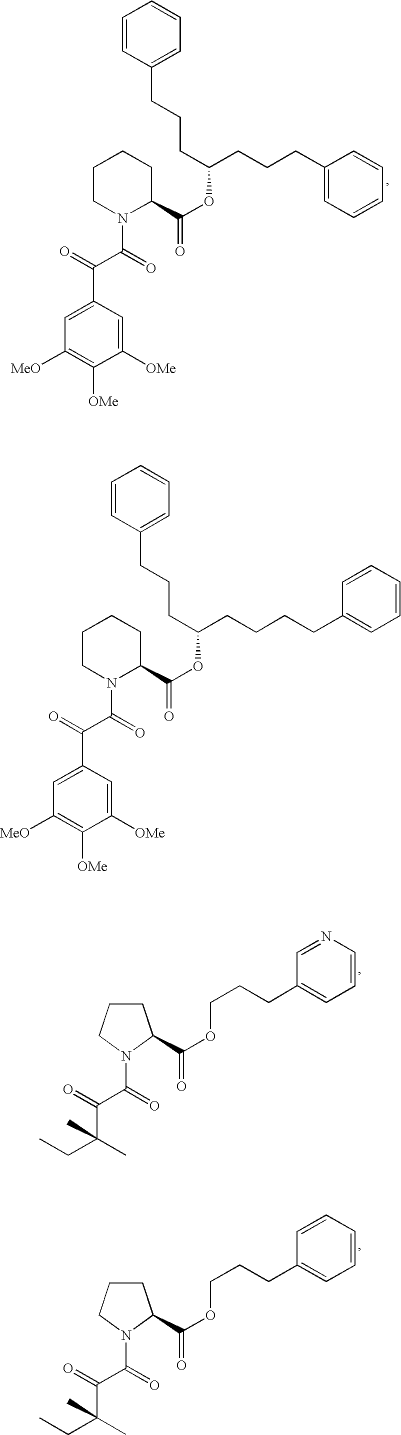 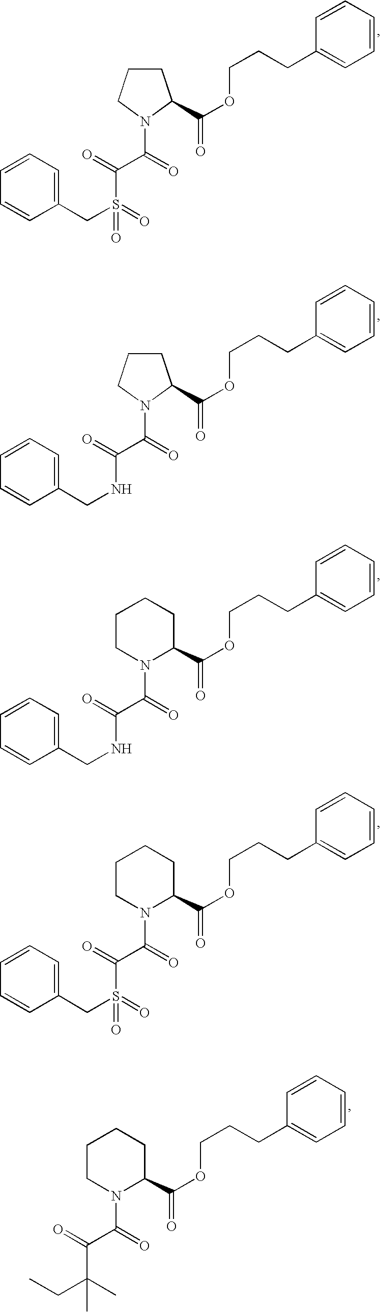 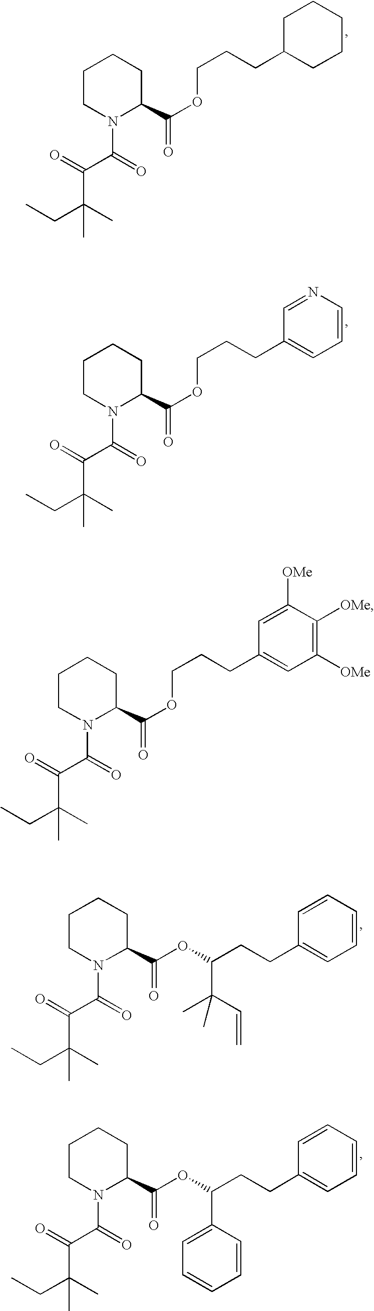 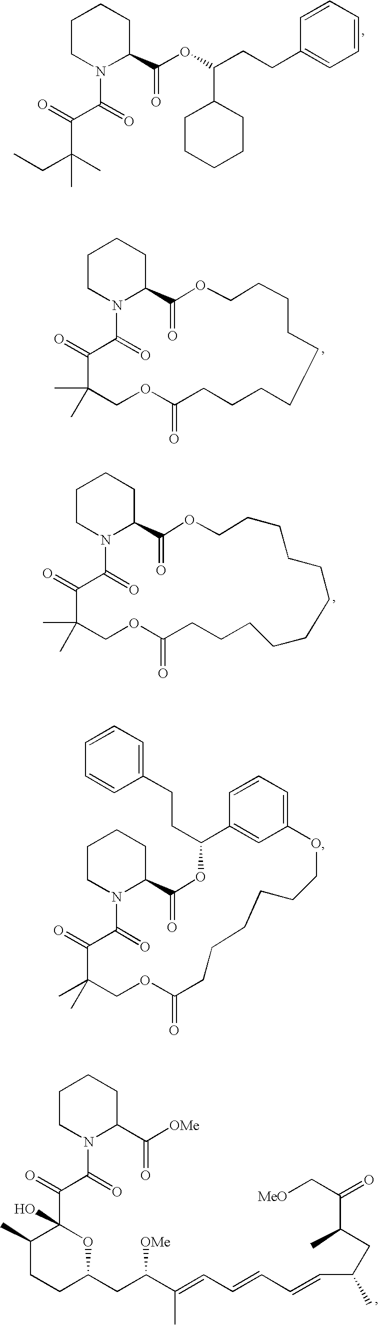 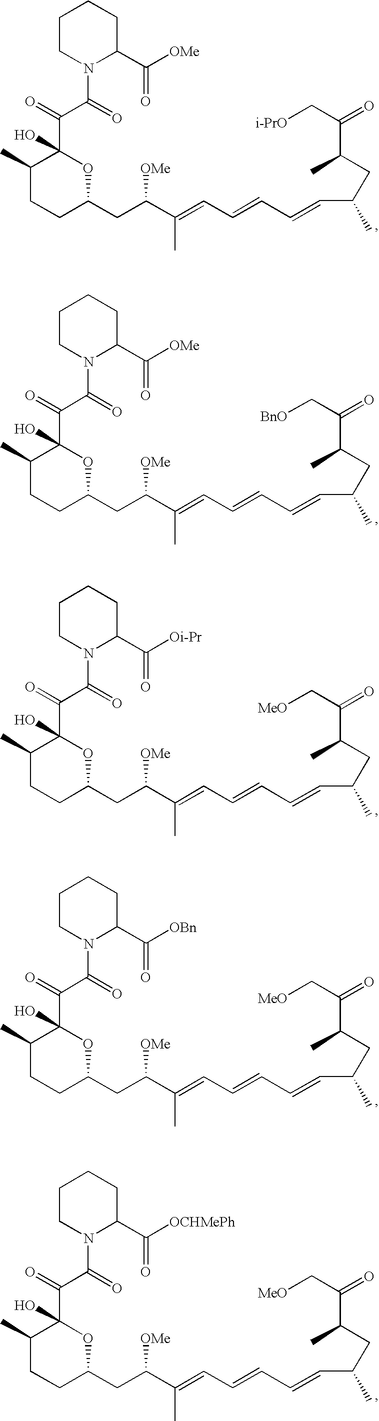 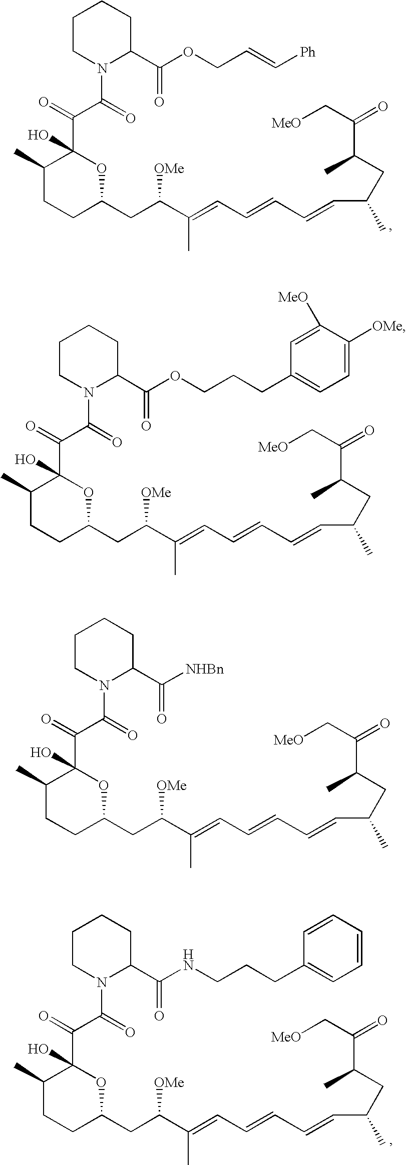 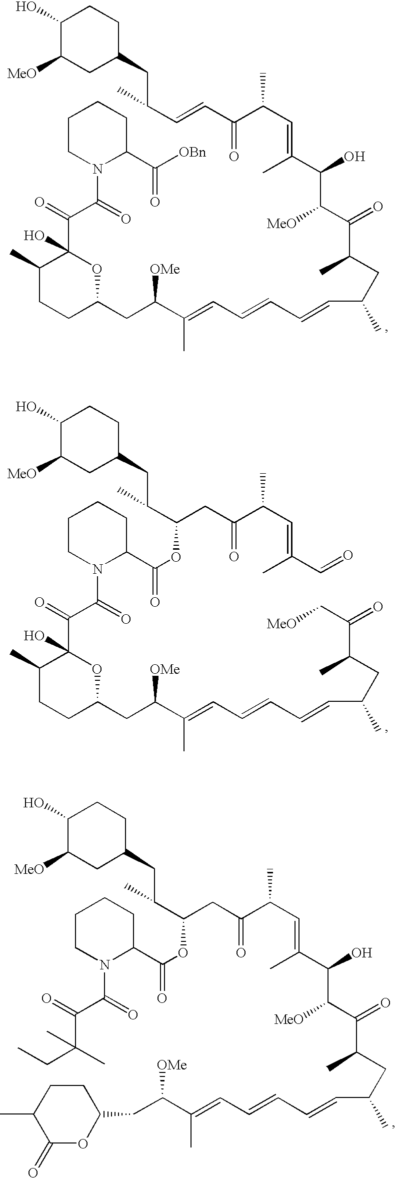 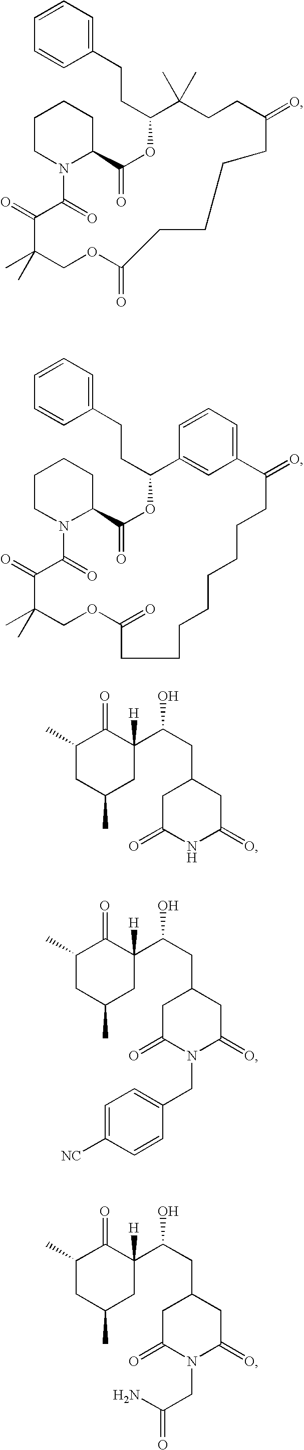 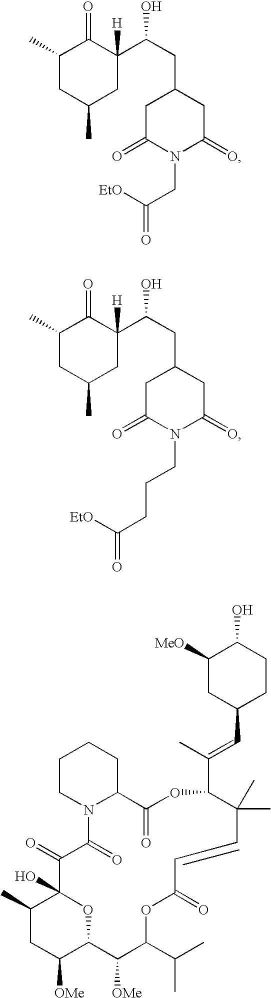
Homoserine Dehydrogenase Inhibitors

RI-331, 5-hydroxy-4-oxonorvaline or HON, is a homoserine dehydrogenase inhibitor having the following structure:

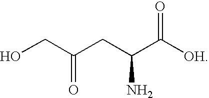

There are numerous known homoserine dehydrogenase inhibitors including, but not limited to, those disclosed by Ejim et al., Bioorg Med Chem. 2004 12(14) 3825-30, hereby incorporated by reference, Jacques et al., Chem Biol. 2003 10(10) 989-95, hereby incorporated by reference, Yamaki et al., J Antibiot. 1992 45(5) 750-5, hereby incorporated by reference, and preferably those of the following formula: 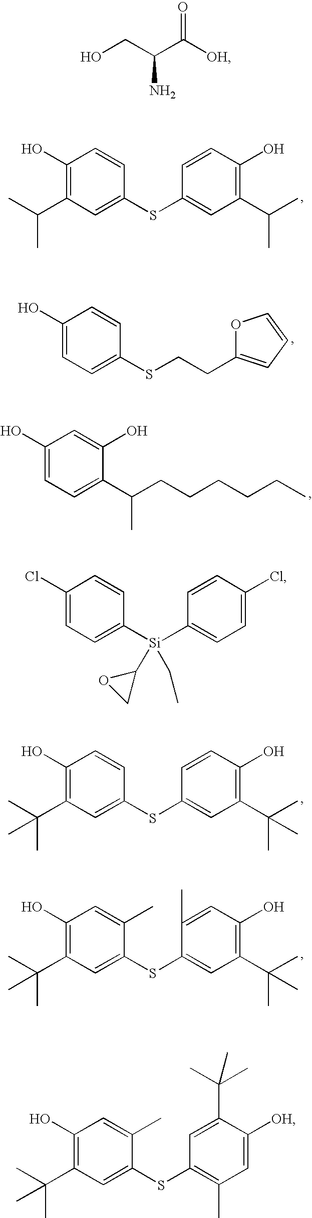 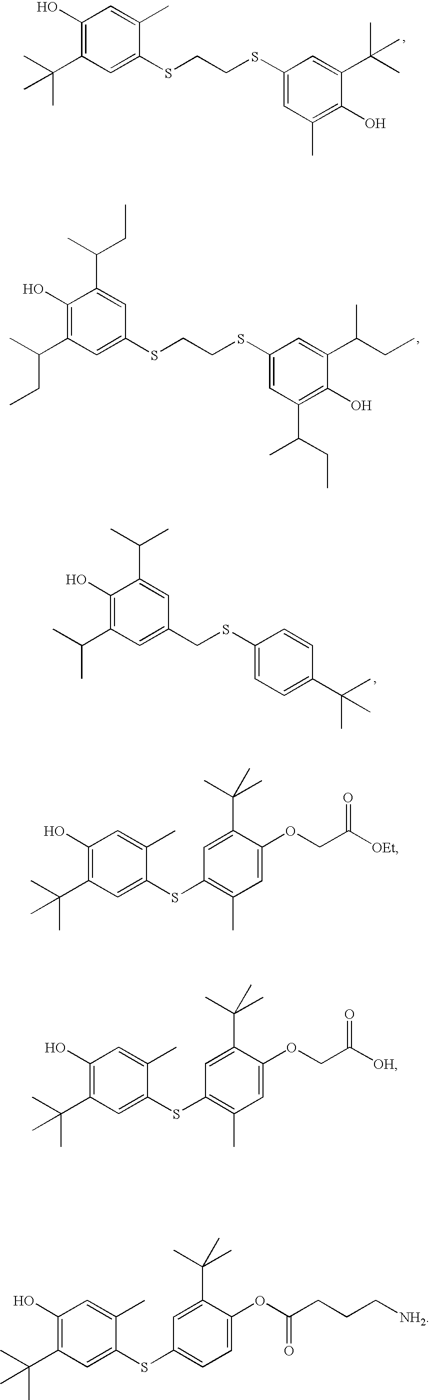
Formulation

Compounds disclosed herein may be formulated together with one or more pharmaceutically acceptable excipients, diluents, carriers, etc. to produce pharmaceutical or veterinary compositions for administration to human or other mammalian recipients, by any of various pharmaceutically approved routes, for the prevention or treatment of pathogenic fungal infection. The compound(s) is administered, typically in the form of a pharmaceutical or veterinary composition containing the compound(s) in admixture with one or more pharmaceutically acceptable carriers, diluents, buffers or other excipients, in an effective amount to prevent or inhibit fungal growth.

Methods and materials for effecting various chemical transformations of are known in the art, as are methods for obtaining compounds that inhibit FKBP12 and homoserine dehydrogenase and various analogs. Many such chemical transformations are disclosed in the patent documents, above, which serve to illustrate the level of skill and knowledge in the art of chemical synthesis and product recovery, purification and formulation which may be applied in practicing the subject invention.

Additionally, it is contemplated that compounds that inhibit FKBP12 and homoserine dehydrogenase and various analogs for use in this invention as well as intermediates for the production of such may be prepared by directed biosynthesis.

Compounds that inhibit FKBP12 and homoserine dehydrogenase and various analogs of this invention may be prepared by one of ordinary skill in this art relying upon methods and materials known in the art as guided by the disclosure presented herein. For instance, methods and materials may be adapted from known methods set forth or referenced in the documents cited above, the full contents of which are incorporated herein by reference. Additional guidance and examples are provided herein by way of illustration and further guidance to the practitioner. It should be understood that the chemist of ordinary skill in this art would be readily able to make modifications to the foregoing, e.g. to add appropriate protecting groups to sensitive moieties during synthesis, followed by removal of the protecting groups when no longer needed or desired, and would be readily capable of determining other synthetic approaches.

Human Immunosuppression Assay Methods

Assays for immunosuppressive activity are known. By way of non-limiting example, immunosuppressive activity (as distinguished from binding activities) may be measured in a mitogenesis assay using human T cells. Such a human T cell proliferation assay is an example of an appropriate in vitro assay for use in determining an AF/IS value for a compound or combination of interest.

In one embodiment, a representative compound can be evaluated in an in vivo test procedure designed to determine the survival time of pinch skin graft from male BALB/c donors transplanted to male C3H(H2K) recipients. The method is adapted from Billingham et al (1951) J. Exp. Biol. 28:385402. Briefly, a pinch skin graft from the donor is grafted on the dorsum of the recipient as an allograft, and an isograft is used as control in the same region. The recipients are treated with either varying concentrations of a test compound(s) intraperitoneally, intravenously or orally. Rapamycin can be used as a test control. Untreated recipients serve as rejection control. The graft is monitored daily and observations are recorded until the graft becomes dry and forms a blackened scab. This is considered as the rejection day. The mean graft survival time (number of days ±S.D.) of the drug treatment group is compared with the control group. An ED50 value can be calculated as the mean ratio of weight compound to weight animal required to produce a mean graft survival time extending to the same period as control graft. The immunosuppressant activities, e.g., immunosuppressant ED50 values of the compound(s) of this invention can be determined via the hemolysin test in mice and by the delayed hypersensitivity test.

An exemplary hemolysin test is that described in Methods in Immunology, edited by D. H. Campbell et al, W. A. Benjamin, New York 1963 pages 172-175, and measures humoral or antibody response. The delayed hypersensitivity test measures the effect of a test compound on the ability of a subject mouse to mount a cell-mediated immune response to the antigen, Mycobacterium tuberculosis H37Ra. The mouse is sensitized to the antigen by subcutaneous administration in the base of the tail. The development of the delayed hypersensitivity response may be measured at any time beginning six days after sensitization but is usually done on the ninth day as follows: The right hind paw is injected with a test compound(s) while the left hind paw (control) receives physiological saline. Both paw volumes are measured after twenty-four hours and significant increase in the volume of the right hind paw is taken as a measure of an effective delayed hypersensitivity response. All compounds are administered by the subcutaneous route. The expression ED50 (mg/kg.) is an expression of the number of milligrams of the test compound(s) per kilogram of body weight administered subcutaneously required to reduce the antibody activity by 50% when compared with a control. In this case, the lower the ED50 value for a compound the more potent an immunosuppressant it is.

The immunosuppressive activity of a compound may also be shown by a graft vs. host reaction (GVHR). In an illustrative embodiment, to induce a GVHR, C57 B 1/6XA/J(F6AF1) male mice are injected intravenously with parental (C57 B 1/6J) spleen and lymph node cells. The compound is then administered orally for 10 days beginning on the day prior to the cell transfer. On the day following the last treatment, the animals are sacrificed, and their spleens excised and weighed. The enlargement of the spleen of the host is a result of a GVHR. To some extent it is the host's own cells which infiltrate and enlarge the spleen although they do this because of the presence of graft cells reacting against the host. The amount of spleen enlargement, splenomegaly, is taken as a measure of the severity of the GVHR. In carrying out the GVHR the animal in the experimental group is injected with parental cells, cells of the same species but of different genotype, which cause a weight increase of the spleen. The animal in the control group is injected with syngeneic cells, genetically identical cells that do not cause a weight increase of the spleen. The effectiveness (ED50) of the compounds administered to the mice in the experimental group is measured by comparing the spleen weight of the untreated and treated GVH animal with that of the syngeneic control. The ED50 value for immunosuppressive activity of compounds can also be measured according to the method of Takatsy et al (1955) Acta. Microbiol. Acad. Sci. Hung., 3:105 or Cottney et al, (1980) Agents and Actions, 10:43.

The immunosuppressive activity of a compound(s) may also be shown by a splenic atrophy test, e.g., a decrease in spleen weight after dosing BDFI mice orally with the drug for seven (7) consecutive days. The mice are sacrificed on the eighth day. The percent decrease in spleen weight is measured for each dosage level.

Assays for Activity Against Pathogenic Fungi

Comparative activity of a combination therapy against a pathogenic fungus, relative to another antifungal such as rapamycin, for use in determining the compounds' AFIS value, is measured directly against the fungal organism, e.g. by microtiter plate adaptation of the NCCLS broth macrodilution method described in Diagn Micro and Infect Diseases 21:129-133 (1995). Antifungal activity can also be determined in whole-animal models of fungal infection. For instance, one may employ the steroid-treated mouse model of pulmonary mucormycosis [Goldani, L. Z. and Sugar, A. M. (1994) J. Antimicrob. Chemother. 33: 369-372]. By way of illustration, in such studies, a number of animals are given no compound, various doses of compounds (and/or combinations with one or more other antifungal agents), or a positive control (e.g. Amphotericin B), respectively, beginning before, at the time of, or subsequent to infection with the fungus. Animals may be treated once every 24 hours with the selected dose of compounds, positive control, or vehicle only. Treatment is continued for a predetermined number of days, e.g. up to ten days. Animals are observed for some time after the treatment period, e.g. for a total of three weeks, with mortality being assessed daily. Models can involve systemic, pulmonary, vaginal and other models of infection with or without other treatments (e.g. treatment with steroids) designed to mimic a human subject susceptible to infection.

To further illustrate, one method for determining the in vivo therapeutic efficacies (ED50, e.g., expressed in mg compound/kg subject), is a rodent model system. For example, a mouse is infected with the fungal pathogen such as by intravenous infection with approximately 10 times the 50% lethal dose of the pathogen (106 C. albicans cells/mouse). Immediately after the fungal infection, compounds are given to the mouse at a predetermined dosed volume. The ED50 is calculated by the method of Van der Waerden (Arch. Exp. Pathol. Pharmakol. 195 389-412, 1940) from the survival rate recorded on 20th day post-infection. Generally, untreated control animals die 7 to 13 days post-infection.

In another illustrative embodiment, C. albicans Wisconsin (C43) and C. tropicalis (C112), grown on Sabouraud dextrose agar (SDA) slants for 48 h at 28° C., are suspended in saline and adjusted to 46% transmission at 550 nm on a spectrophotometer. The inoculum is further adjusted by hemacytometer and confirmed by plate counts to be approximately 1 or 5×10⁷ CFU/ml. CF-1 mice (white, male, ca. 20 g, Harlan Sprague Dawley, Inc., Indianapolis, Ind.) are infected by injection 1 or 5×10⁶ CFU into the tail vein. Antifungal agents are administered intravenously or subcutaneously in ethanol:water (10:90), 4 h post infection and once daily thereafter for 3 or 4 more days. Survival is monitored daily. The ED50 can be defined as that dose which allows for 50% survival of mice.

Pharmaceutical Compositions

In a preferred embodiment, the present invention pertains to combination therapies based on compounds that inhibit of the activity or expression of proteins FKBP12 and homoserine dehydrogenase, preferably a non-immunosuppressive FKBP12 inhibitor as described above and a pharmaceutically acceptable carrier forming pharmaceutical composition. It is preferred that the compounds will be present in an effective amount to prevent or treat pathogenic fungal infection when administered to a subject in need thereof. The pharmaceutical composition also can contain other additives that do not detrimentally affect the ability of the compounds to perform its intended antifungal function, numerous examples of which are known in the art.

Compounds that inhibit the activity or expression of proteins FKBP12 and homoserine dehydrogenase can exist in free form or, where appropriate or desired, in the form of a pharmaceutically acceptable derivative, including an ester, salt, etc. Pharmaceutically acceptable salts and their preparation are well known to those of skill in the art. The pharmaceutically acceptable salts of such compounds include the conventional non-toxic salts or the quaternary ammonium salts of such compounds that are formed, for example, from inorganic or organic acids of bases. The compounds of the invention may form hydrates or solvates. It is known to those of skill in the art that charged compounds form hydrated species when lyophilized with water, or form solvated species when concentrated in a solution with an appropriate organic solvent.

The amount of compounds that will be effective in the treatment or prevention of a particular fungal pathogen will depend in part on the characteristics of the fungus and the extent of infection, and can be determined by standard clinical techniques. In vitro or in vivo assays may optionally be employed to help identify optimal dosage ranges. Effective doses may be extrapolated from dose-response curves derived from in vitro analysis or preferably from animal models. The precise dosage level should be determined by the attending physician or other health care provider and will depend upon well known factors, including route of administration, and the age, body weight, sex and general health of the individual; the nature, severity and clinical stage of the infection; the use (or not) of concomitant therapies.

The effective dose of the compounds will typically be in the range of about 0.01 to about 50 mg/kgs, preferably about 0.1 to about 10 mg/kg of mammalian body weight per day, administered in single or multiple doses. Generally, the compound may be administered to patients in need of such treatment in a daily dose range of about 1 to about 2000 mg per patient. In embodiments in which the compound can result in some residual immunosuppressive effects, it is preferred that the dose administered be below that associated with therapeutic immunosuppressive effects.

In one embodiment, the composition of combined inhibitors can be a liquid solution, suspension, emulsion, tablet, pill, capsule, sustained release formulation, cream, foam, or powder. The composition can be formulated as a suppository, e.g. with traditional binders and carriers such as triglycerides. Oral formulation can include standard carriers such as pharmaceutical grades of mannitol, lactose, starch, magnesium stearate, sodium saccharine, cellulose, magnesium carbonate, etc.

Formulation may involve mixing, granulating and compressing or dissolving the ingredients as appropriate to the desired preparation. The pharmaceutical carrier employed may be, for example, either a solid or liquid. Illustrative solid carriers include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid that is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins. Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellant. Liquid pharmaceutical compositions that are sterile solutions or suspensions can be utilized by, for example, intramuscular, intraperitoneal or subcutaneous injection. Sterile solutions can also be administered intravenously. The compound can also be administered orally either in liquid or solid composition form. The carrier or excipient may include time delay material well known to the art, such as glyceryl monostearate or glyceryl distearate along or with a wax, ethylcellulose, hydroxypropylmethyl-cellulose, methylmethacrylate and the like. When formulated for oral administration, 0.01% Tween 80 in PHOSAL PG-50 (phospholipid concentrate with 1,2-propylene glycol, A. Nattermann & Cie. GmbH) has been recognized as providing an acceptable oral formulation for rapamycin, and may be adapted to formulations for various FKBP12 and homoserine dehydrogenase inhibitors. A wide variety of pharmaceutical forms can be employed. If a solid carrier is used, the preparation can be tableted, placed in a hard gelatin capsule in powder or pellet form or in the form of a troche or lozenge. The amount of solid carrier will vary widely but preferably will be from about 25 mg to about 1 g. If a liquid carrier is used, the preparation will be in the form of a syrup, emulsion, soft gelatin capsule, sterile injectable solution or suspension in an ampule or vial or nonaqueous liquid suspension. To obtain a stable water-soluble dosage form, a pharmaceutically acceptable salt of a compound(s) may be dissolved in an aqueous solution of an organic or inorganic acid, such as a 0.3 M solution of succinic acid or citric acid. Alternatively, acidic derivatives can be dissolved in suitable basic solutions. If a soluble salt form is not available, the compound is dissolved in a suitable cosolvent or combinations thereof. Examples of such suitable cosolvents include, but are not limited to, alcohol, propylene glycol, polyethylene glycol 300, polysorbate 80, glycerin, polyoxyethylated fatty acids, fatty alcohols or glycerin hydroxy fatty acids esters and the like in concentrations ranging from 0-60% of the total volume.

Various delivery systems are known and can be used to administer the compounds, or the various formulations thereof, including tablets, capsules, injectable solutions, encapsulation in liposomes, microparticles, microcapsules, etc. Methods of introduction include but are not limited to dermal, intradermal, intramuscular, intraperitoneal, intravenous, subcutaneous, intranasal, intravaginal, extravaginal, pulmonary, epidural, ocular and (as is usually preferred) oral routes. Dermal administration can be in the form of a topical solution, cream, foam, or the like, and can involve any skin surface, including the feet and toes.

The compounds may be administered by any convenient or otherwise appropriate route, for example by infusion or bolus injection, by absorption through epithelial or mucocutaneous linings (e.g., oral mucosa, rectal and intestinal mucosa, etc.) and may be administered together with other biologically active agents. Administration can be systemic or local. For treatment or prophylaxis of nasal, bronchial or pulmonary infection, preferred routes of administration are oral, nasal or via a bronchial aerosol or nebulizer. In certain embodiments, it may be desirable to administer the compound locally to an area in need of treatment; this may be achieved by, for example, and not by way of limitation, local infusion during surgery, topical application, by injection, by means of a catheter, by means of a suppository, or by means of a skin patch or implant, said implant being of a porous, non-porous, or gelatinous material, including membranes, such as silastic membranes, or fibers. In a specific embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the side of the injection. Generally, the ingredients are supplied either separately or mixed together in unit dosage form, for example, as a lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Administration to an individual of an effective amount of the compound can also be accomplished topically by administering the compound(s) directly to the affected area of the skin of the individual. For this purpose, the compound is administered or applied in a composition including a pharmacologically acceptable topical carrier, such as a gel, an ointment, a lotion, or a cream, which includes, without limitation, such carriers as water, glycerol, alcohol, propylene glycol, fatty alcohols, triglycerides, fatty acid esters, or mineral oils. Other topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents may be added as necessary. Percutaneous penetration enhancers such as Azone may also be included. In addition, in certain instances, it is expected that the compound may be disposed within devices placed upon, in, or under the skin. Such devices include patches, implants, and injections that release the compound into the skin, by either passive or active release mechanisms. Materials and methods for producing the various formulations are well known in the art and may be adapted for practicing the subject invention.

In a preferred embodiment, the compounds are formulated for oral administration, as for example in the form of a solid tablet, pill, capsule (including gel capsules), caplet or the like (collectively hereinafter “tablet”) or an aqueous solution or suspension. In a preferred embodiment of the tablet form of the compounds, the tablets are preferably formulated such that the amount of compounds provided in 20 tablets, if taken together, would provide a dose of at least the median effective dose (ED50), e.g., the dose at which at least 50% of individuals exhibited the quantal effect of inhibition of fungal cell proliferation in vivo (or a statistically significant reduction in infection). More preferably, the tablets are formulated such that the total amount of compounds provided in 10, 5, 2 or 1 tablets would provide at least an ED50 dose to a patient (human or non-human mammal). In other embodiments, the amount of compounds provided in 20, 10, 5 or 2 tablets taken in a 24 hour time period would provide a dosage regimen providing, on average, a mean plasma level of the compounds of at least the EC50 concentration (the concentration for 50% of maximal effect of, e.g., inhibiting fungal cell proliferation or statistically reducing the level fungal infection). An ED50 dose, for a human, is based on a body weight of from 10 lbs to 250 lbs, though more preferably for an adult in the range of 100 to 250 lbs.

In preferred embodiments, a single dose of tablets (1-20 tablets) provides about 0.25 mg to 1250 mg of the compounds.

Likewise, the compounds can be formulated for parenteral administration, as for example, for subcutaneous, intramuscular or intravenous injection, e.g., the compounds can be provided in a sterile solution or suspension (collectively hereinafter “injectable solution”). The injectable solution is preferably formulated such that the amount of compounds provided in a 200 cc bolus injection would provide a dose of at least the median effective dose. More preferably, the injectable solution is formulated such that the total amount of compounds provided in 100, 50, 25, 10, 5, 2.5, or 1 cc injections would provide an ED50 dose to a patient. In other embodiments, the amount of compounds provided in a total volume of 100 cc to be injected at least twice in a 24 hour time period would provide a dosage regimen providing, on average, a mean plasma level of the compounds of at least the EC50 concentration. In preferred embodiments, a single dose injection provides about 0.25 mg to 1250 mg of compound. For continuous intravenous infusion, e.g., drip or push, the compounds can be provided in a sterile dilute solution or suspension (collectively hereinafter “i.v. injectable solution”). The i.v. injectable solution is preferably formulated such that the amount of compounds provided in a 1 L solution would provide a dose, if administered over 15 minutes or less, of at least the median effective dose. More preferably, the i.v. injectable solution is formulated such that the total amount of compound provided in 1 L solution administered over 60, 90, 120 or 240 minutes would provide an ED50 dose to a patient. In preferred embodiments, a single i.v. “bag” provides about 0.25 mg to 5000 mg of compounds per liter i.v. solution, more preferably 0.25 mg to 2500 mg, and even more preferably 0.25 mg to 1250 mg. As discussed above, the preferred compound pharmaceutical preparation, whether for injection or oral delivery (or other route of administration), would provide a dose less than the ED50 for immunosuppression in the host, more preferably at least 1 order of magnitude less, more preferably at least 2, 3 or 4 orders magnitude less. That is, the compounds are dosed in sub-immunosuppressive amounts.

Combination products which contain at least one compounds that inhibit of the activity or expression of proteins FKBP12 and at least one compound that inhibits homoserine dehydrogenase are contemplates as described above and an immunosuppressant agent such as FK506, rapamycin, cyclosporin A or the like; an antifungal agent such as amphotericin B, an analog or derivative thereof or another polyene macrolide antibiotic; flucytosine; griseofulvin; imidazole or triazole antifungal agent such as, e.g., clotrimazole, miconazole, ketoconazole, econazole, butoconazole, oxiconazole, terconazole, itraconazole or fluconazole, or another one or more antifungal agents such as are mentioned previously; one or more antibacterial agents; one or more antiviral agents or other agents for treating a patient infected with HIV such as are discussed above; or one or more immune response modifiers, may be formulated as described above or may be formulated based on formulation materials and methods used in connection with the other agent(s) in the combination. In the case of combination with one or more other antifungal agents, one or more active ingredients may be provided in reduced amounts (relative to amounts used in stand-alone products) by virtue of the combination. Alternatively, the compounds that inhibit of the activity or expression of proteins FKBP12 and homoserine dehydrogenase and combination agent(s) may be separately formulated.

Packaging

The present invention also pertains to packaged compounds, preferably packaged non-immunosuppressive combination therapy, as described above, packaged with instructions for administering the compounds as an antifungal agent without causing a theraputic immunosuppressive effects within a human subject. In some embodiments, the non-immunosuppressive FKBP12 is a member of one of the preferred subsets of compounds described above. The compounds can be packaged alone with the instructions or can be packaged with another ingredient or additive, e.g., one or more of the ingredients of the pharmaceutical compositions of the invention. The package can contain one or more containers filled with one or more of the ingredients of the pharmaceutical compositions of the invention. Optionally associated with such container(s) can be a notice in the form prescribed by a governmental agency regulating the manufacture, use or sale of pharmaceutical or biological products, which notice reflects approval by the agency of manufacture, use or sale for human administration.

Methods of Treating Fungal Infections

One method involves administering at least one compound that inhibits the activity or expression of FKBP12 and at least one compound that inhibits the activity or expression homoserine dehydrogenase, preferably a non-immunosuppressive FKBP12 inhibitor, as described above, to a human subject such that the fungal infection is treated or prevented without inducing an therapeutic immunosuppressive effect in the human subject. In certain embodiments the compounds are administered in conjunction with administration of another antifungal agent such as amphotericin B, or an imidazole or triazole agent such as those mentioned previously.

The pathogenic fungal infection may be topical, e.g., caused by, among other organisms, species of Candida, Trichophyton, Microsporum or Epidermophyton or mucosal, e.g., caused by Candida albicans (e.g. thrush and vaginal candidiasis). The infection may be systemic, e.g., caused by Candida albicans, Cryptococcus neoformans, Aspergillus fumigatus, Coccidioides, Paracoccidioides, Histoplasma or Blastomyces species. The infection may also involve eumycotic mycetoma, chromoblastomycosis, cryptococcal meningitis or phycomycosis.

In one embodiment, the method for treating or preventing a pathogenic fungal infection involves the administration of one or more compounds that inhibit the activity or expression of proteins FKBP12 in conjunction with (whether simultaneously or separately) one or more compounds that inhibit the activity or expression of homoserine dehydrogenase, preferably a non-immunosuppressive FKBP12 inhibitor, to an immunocompromised subject. Preferably, the drug is administered to the immunocompromised subject without inducing a further therapeutic immunosuppressive effect. Examples of such subjects include subjects infected with HIV including patients with AIDS; immunosuppressed patients who have received, or are being treated with immunosuppressant therapy in preparation for receiving, an organ or tissue transplant, including e.g. bone marrow transplants; subjects having an autoimmune disorder or disease, e.g., diabetes, MS, myasthenia gravis, systemic lupus erythematosus or rheumatoid arthritis; and cancer patients who are immunosuppressed as a result of cancer chemotherapy or radiation therapy. It will often be preferred that the antifungal therapy spare any remaining immune function of the patient and that the compounds be given in a sub-immunosuppressive dose or regimen. Where compounds are administered, it will be preferred in some such cases that the compounds be a non-immunosuppressive FKBP12 inhibitor. The compounds may be administered in combination with one or more other antifungal agents to provide an antifungal therapy with a reduced dosage of one or more of the active ingredients as well as in the other combinations disclosed herein.

In another embodiment, the invention provides a method for treating or preventing a pathogenic fungal infection selected from the group consisting of Candida species including C. albicans, C. tropicalis, C. kefyr, C. krusei and C. galbrata; Aspergillus species including A. fumigatus and A. flavus; Cryptococcus neoformans; Blastomyces species including Blastomyces dermatitidis; Pneumocystis carinii; Coccidioides immitis; Basidiobolus ranarum; Conidiobolus species; Histoplasma capsulatum; Rhizopus species including R. oryzae and R. microsporus; Cunninghamella species; Rhizomucor species; Paracoccidioides brasiliensis; Pseudallescheria boydii; Rhinosporidium seeberi; and Sporothrix schenckii. Again, the method preferably involves administering a non-immunosuppressive FKBP12 inhibitor to a patient in need thereof such that the fungal infection is treated or prevented without inducing a therapeutic immunosuppressive effect.

In a further embodiment, the invention provides a method for treating or preventing a pathogenic fungal infection which is resistant to other antifungal therapy, including pathogenic fungal infections which are resistant to one or more antifungal agents mentioned elsewhere herein such as amphotericin B, flucytosine, one of the imidazoles or triazoles (including e.g. fluconazole, ketoconazole, itraconazole and the other previously mentioned examples). The method involves administering to the patient one or more compounds that inhibit the activity or expression of proteins FKBP12 and homoserine dehydrogenase, preferably a non-immunosuppressive FKBP12 inhibitor, in an amount and dosing regimen such that a fungal infection resistant to another antifungal therapy in the subject is treated or prevented.

In a further embodiment, the invention provides a method for treating or preventing a pathogenic fungal infection in a patient who is allergic to, intolerant of or not responsive to another antifungal therapy or in whom the use of other antifungal agents is otherwise contra-indicated, including one or more other antifungal agents mentioned elsewhere herein such as amphotericin B, flucytosine, one of the imidazoles or triazoles (including e.g. fluconazole, ketoconazole, itraconazole and the other previously mentioned examples). The method involves administering to such patient one or more compounds that inhibit the activity or expression of proteins FKBP12 and homoserine dehydrogenase, preferably a non-immunosuppressive FKBP12 inhibitor, in an amount such that a fungal infection is treated or prevented.

Materials and Methods

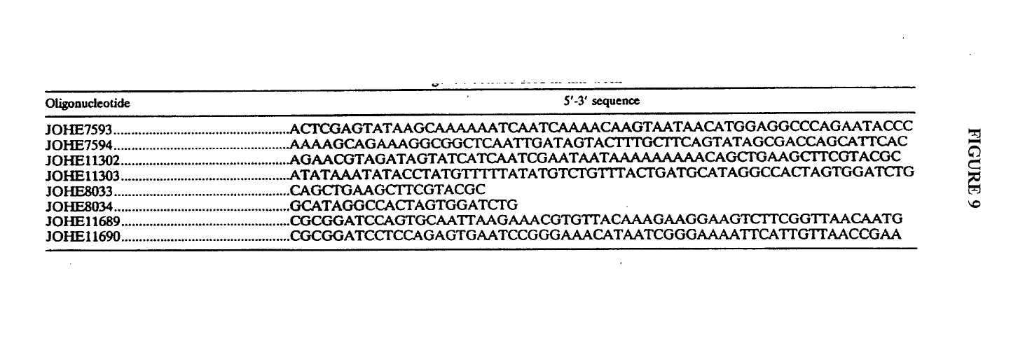

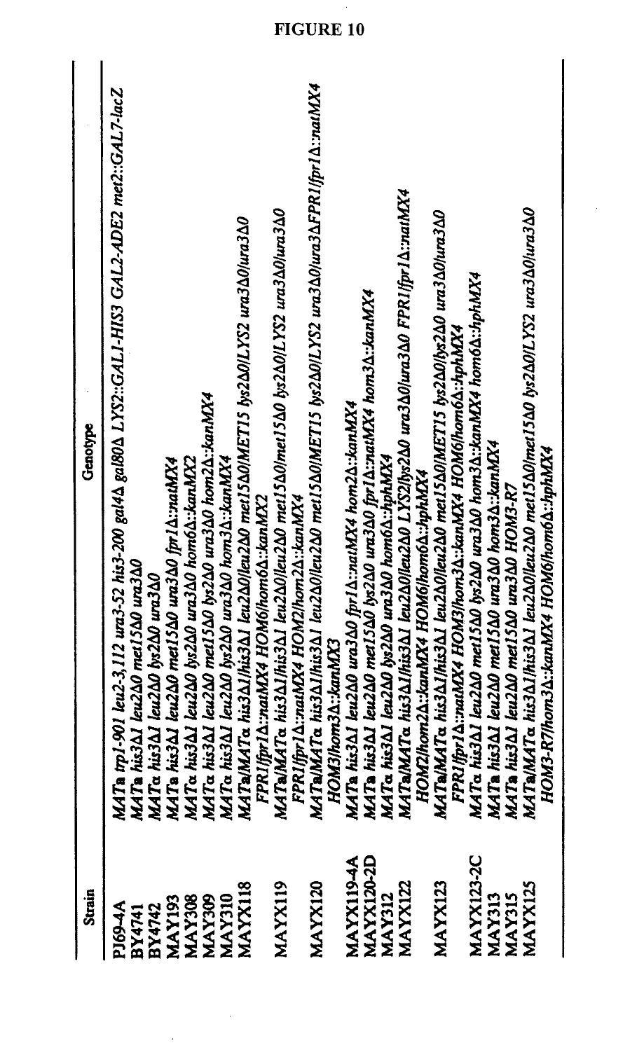

2 μm plasmid pSEY8 were described in Heitman et al., Proc. Natl. Acad. Sci 1991 88, 1948-1952 and Jungmann et al., EMBO 1993 12, 5051-5056, hereby incorporated by reference. Centromere-based plasmid pMA-HOM6, expressing wild-type HOM6 gene, was obtained by gap repair. With this aim, a synthetic, minimal HOM6 gene, allele was first obtained by PCR using 3′complementary primer pairs JOHE11689 and JOHE11690 (FIG. 9), digested with BamHI and cloned into the BamHI site of the vector pRS316. The resulting plasmid, pMA-hom6Δ, contains an insert consisting of two 40-bp-long segments, corresponding to sequences found 300 bp upstream and downstream of the HOM6 open reading frame, respectively, and flanking a unique HpaI restriction site. Plasmid pMA-HOM6 was rescued from yeast strain BY4741 transformed with plasmid pMA-hom6Δ, previously linearized by HpaI digestion. Two-hybrid expression vectors, pGBT9 and pGAD424 were as described in Bartel et al., Cellular interactions in development: a practical approach 1993, 153-179. Plasmids pGBT-Fpr1 expresses Fpr1 fused to the C terminus of the Ga14 DNA binding domain. Plasmids pGBT9-AK and pGAD424 express wild-type AK fused to the C terminus of the Ga14 DNA binding domain or Ga15 activation domain, respectively. Plasmid pMACR7 expresses the mutant HOM3-R7 allele. Plasmid pGBT9-AK(E282D) expresses the AK^{E282D} fused to the C terminus of the Ga14 DNA-binding domain and was constructed by cloning in pGBT9-AK the SwaI-NdeI restriction fragment of pMACR7 containing the HOM3-R7 GAA₈₄₆ GAT mutation determining E282D amino acid substitution. Plasmid pGAD424-AK(E282D) expresses AK^{E282D} fused to the C terminus of the Ga14 activation domain, was constructed by cloning the smaller BamHI-EcoRI fragment of pGAD424-AK(E282D) in to plasmid pGAD424, previously digested with enzymes. Centromere-based plasmids pFP101 and pFP102, expressing wild-type Fpr1 and the active-site mutant Fpr1^{F43Y}, respectively, were as described in Hemenway et al., J. Biol. Chem. 1996 272, 18527-18534, hereby incorporated by reference. Centromere-based vector YCplac111, was used as described in Gietz and Sugino Gene 1988 74, 527-534, hereby incorporated by reference.

With the exception of two-hybrid host strain PJ69-4A, all of the yeast strains used are derivative of the isogenic S288C-derived strain BY4741, BY4742, or BY4743. Stain MAY193 was obtained from strain BY4741 by disruption of the FPR1 gene with nourseothricin resistance natMX4 module from plasmid pAG25, PCR amplified with primers JOHE7593 and JOHE7594. Strain MAY308 was obtained from strain BY4742 by disruption of the HOM6 gene with G418 resistance KanMX2 module from plasmid pFA6-KanMX2, PCR amplified with primers JOHE11302 and JOHE11303. Strains MAY309, MAY310, MAY313 were obtained as meiotic products of the corresponding hom2Δ/hom2Δ and home3Δ/hom3Δ homozygous diploid strains. Diploid strains MAYX118, MAYX119, MAYX120 were obtained by crossing strains MAY193 with strains MAY308, MAY309, and MAY310, respectively. Strains MAYX119-4A and MAYX120-2D were obtained as meiotic products of strains MAYX119 and MAYX120. Strain MAY312 was obtained from strain MAY308 by substitution of the kanMX2 module in the hom6Δ::kanMX2 allele with the hygromycin B resistance hphMX4 module from plasmid pAG32 which had been PCR amplified with primers JOHE8033 and JOHE8034. Strains MAYX122 and MAYX123 were obtained by crossing strain MAY312 with strains MAYX119-4A and MAYX120-2D. Strain MAYX123-2C was obtained as a meiotic product of strain MAY123. Strain MAY315 was obtained from strain MAY313 by replacing the hom3Δ::kanMX4 allele of the strain with the HOM3-R7 allele carried in the HpaI-XbaI fragment of plasmid pMACR7. Strain MAYX125 was obtained by crossing strain MAYX123-2C with MAY315.

Growth media for S. Cerevisiae (synthetic minimal medium [YNB], synthetic complete medium [SC] and rich complex medium [YPD]) are described in Sherman F., Methods Enzymol. 2002 350, 3-41, hereby incorporated by reference. Affinity purification of the His6-Frp1 protein and Fpr1 affinity chromatography was performed as disclosed in Cardenas et al., EMBO J., 14, 5892-5907, hereby incorporated by reference.

Yeast two-hybrid host strain PJ69-4A was cotransformed with plasmids expressing the Ga14 DNA binding-domain (BD) and Ga14 activation domain (AD) fusion proteins. The transformants were grown in Siluid SD medium supplemented with adenine, uracil, methionine, and histidine or in the same medium with the 1 g of L-threonine/liter or with 10 mg of FK506/liter, and induction of the lacZ reporter gene was measured as beta-galactosidase activity as disclosed in Cardenas et al., EMBO J., 14, 5892-5907.

For Western blot analysis of expression of AK and Fpr1, yeast strains expressing these proteins were cultured in liquid YPD medium. Whole-cell protein extracts were prepared by glass bead disruption in lysis buffer A (20 mM HEPES [pH 7.4], 20 mM KCl, 0.5 mM EDTA, and a cocktail of protease inhibitors consisting of 0.5 mM phenylmethylsulfonyl fluoride, 1 μg of pepstatin mL-1, 1 mM benzamidine, and 0.001% aprotinin), using a FastPrep instrument (FP120; Bio 101 Savant)/Proteins were resolved by sodium difluoride membrane (Immun-Blot; Bio-Rad), probed with rabbit polyclonal antisera against Fpr1 or with rabbit polyclonal antisera against aspartokinase. Reactions were detected with ECL (Amersham Biosciences).

AK partial purification was as described Farfan et al., Biotechnol. Bioeng. 1996, 49, 667-674. AK activity was measured with an enzymatic assay that couples ADP formation with NADH depletion, using pyruvate kinase/lactate dehydrogenase system.

### EXAMPLE 1

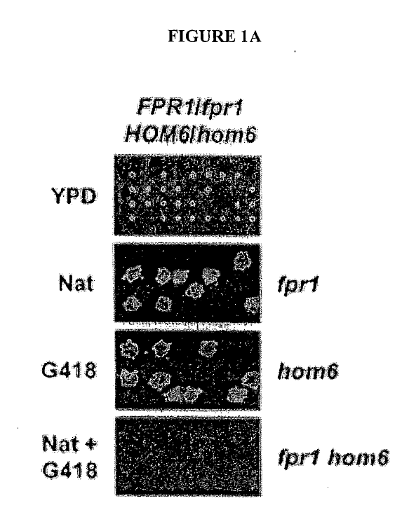

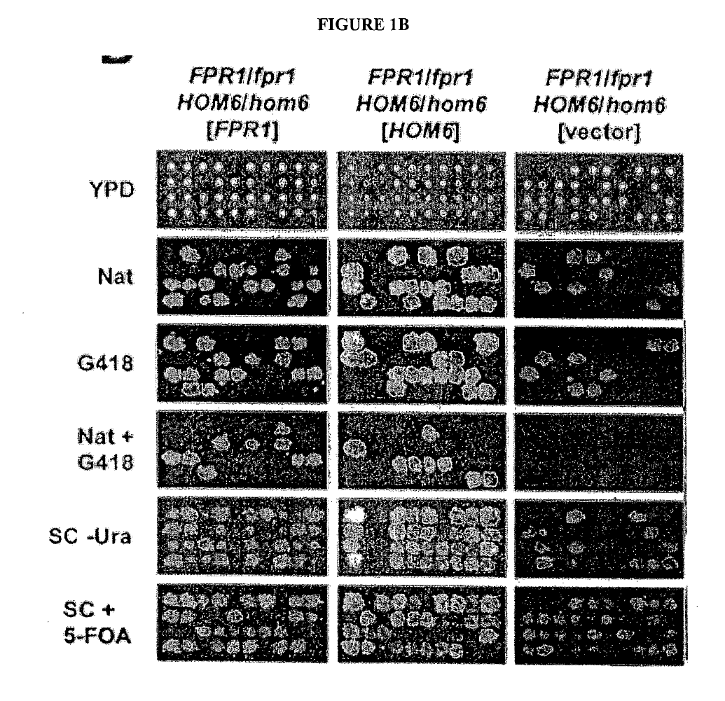

The synthetic lethal interaction between the fpr1Δ and hom6Δ mutations was tested by triad analysis. Fpr1Δ and hom6Δ single mutants were constructed by replacing the entire FPR1 and HOM6 open reading frames with nourseothricin and G418 resistance modules, respectively. The resulting fpr1Δ::nat and hom6Δ::kan strains were crossed to obtain an FPR1/fpr1Δ::nat HOM6/hom6Δ::kan doubly heterozygous mutant diploid strain. As shown in FIG. 1A, this diploid strain sporulated to produce haploid meiotic progeny that were resistant to nourseothricin (Nat^{r}) or to G418(G418^{r}) but not to both drugs indicating that fpr1Δhom6Δ double mutant is inviable. Microscopic observation of meiotic products with an inferred fpr1Δhom6Δ genotype (deduced form the genotype of their tetrad siblings) revealed that these spores germinate and undergo a limited number of cell divisions prior to growth cessation. Neither the fpr1Δ mutation nor the hom6Δ mutation exhibited synthetic lethality, with the met15Δ0 or lys2Δ0 mutation also segregating in this cross.

Viability of fpr1Δ hom6Δ double mutants was rescued by ectopic expression of plasmid-borne copies of FPR1 or FPR6, indicating that lethality of the double mutant is attributable to deficiencies in FPR1 and HOM6 encoded functions. The FPR1/fpr1Δ::nat HOM6/hom6Δ::kan diploid strain was transformed with URA3-selectable plasmids expressing either the wild-type FPR1 gene or the HOM6 gene, and the resulting strains produced Ura⁺ Nat^{r} G418^{r} segregants. All Ura⁺ Nat^{r} G418^{r} meiotic segregants were sensitive to counterselection of the URA3 plasmid-borne marker with 5-fluoroorotic acid, indicating that the FRP1 and HOM6 expressing plasmids are required for viability. Sporulation of the FPR1/fpr1Δ::nat HOM6/hom6Δ::kan diploid strain transformed with a URA3 control vector failed to produce any viable fpr1hom6 (Ura⁺ Nat^{r} G418^{r}) meiotic products. (FIGS. 1A and 1B)

### EXAMPLE 2

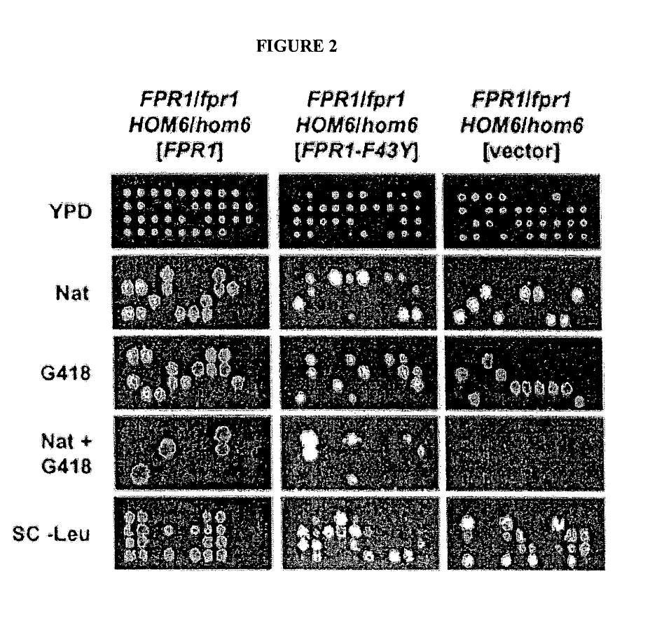

Expression of an FKBP12 mutant protein with reduced prolyl-isomerase activity restores viability of fpr1Δhom6Δdouble mutants. The FPR1/fpr1Δ::nat HOM6/hom6Δ::kan diploid strain was transformed with a centromere-based LEU2 Plasmid expressing the Fpr1^{F43Y} mutant. We noted that the growth rate of fpr1Δhom6Δ colonies expressing Fpr1^{Y43Y} was lower than that of those expressing wild-type Fpr1 from the same LEU2 vector in a control experiment, believed to be attributable to reduced expression of the Fpr1^{Y43Y} mutant. (FIG. 2)

### EXAMPLE 3

One prediction was that fpr1Δhom3Δ and fpr1Δhom2Δ double mutants would exhibit a lethal phenotype, similar to that observed for fpr1Δhom6Δ double mutants. G418-resistant hom3Δ::kan and hom2Δ::kan constructed single mutants were mated with fpr1Δ::nat strains providing FPR1/fpr1Δ::nat HOM3/hom3Δ::kan and FPR1/fpr1Δ::nat HOM2/hom2Δ::kan diploid strains. As shown in FIG. 3B, sporulation of these strains produced viable Nat^{r} and G418^{r} spores that exhibited no growth defect, indicating that the fpr1Δhom3Δ and fpr1Δhom3Δ double mutants are viable and therefore capable of efficient threonine uptake. Thus, the synthetic lethal interaction observed between hom6 and fpr1 is gene specific and is not observed with other hom mutations.

### EXAMPLE 4

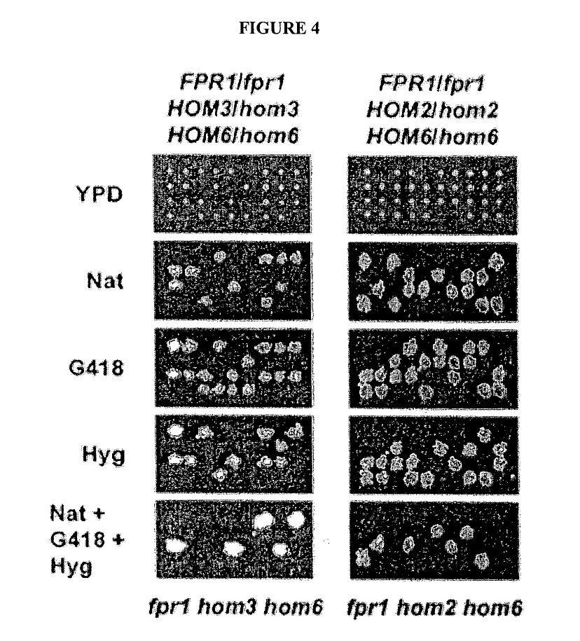

A deletion of HOM3 or HOM2 suppresses lethality of fpr1Δhom6Δ double mutants. If lethal phenotype fpr1Δhom6Δ double mutants accumulate toxic levels of aspartate beta-semialdehyde, then mutation of enzymes earlier in the synthetic pathway would block beta-semialdehyde formation and restore viability of fpr1Δhom6Δ mutant strains. Hom3Δ and hom 2Δ mutations suppressed lethality of the fpr1Δhom6Δ double mutants. Fpr1Δhom3Δ and fpr1Δhom2Δ double mutant strains were crossed with a hom6Δ::hph mutant, and FPR1/fpr1 HOM3/hom HOM6/hom6 and FPR1/fpr1 HOM2/hom2 HOM6/hom6 diploid strains heterozygous at three loci were isolated. Sporulation of these diploids produced no viable fpr1hom6 double mutants (Ura⁺ Nat^{r} G418^{r}) confirming synthetic lethality of fpr1 and hom6Δ mutations in these crosses. In contrast viable fpr1Δhom3Δhom6Δ and fpr1Δhom2Δhom6Δ triple mutant strains (Nat^{r} G418^{r} Hyg^{r}) were readily isolated and the growth of these triple mutants was indistinguishable from that of wild type supporting a model in which aspartate beta-semialdehyde accumulation is toxic and results in a lethal phenotype observed in fpr1Δhom6Δ mutants. (FIG. 4)

### EXAMPLE 5

Deletion of HOM6 is deleterious to strains expressing and aspartokinase resistant to feedback inhibition. Flux through the homoserine biosynthetic pathway is governed through feedback inhibition of aspartokinase by threonine. Mutations that render aspartokinase resistant to feedback inhibition lead to threonine overproduction. Aspartokinase was identified as a binding partner of FKBP12, suggesting FKBP12 regulates aspartokinase inhibition by threonine. If loss of FKBP12 function results in deregulation of aspartokinase activity, deletion of FPR1 in a hom6Δ mutant could lead to aspartate beta-semialdehyde accumulation. Additionally, a HOM3 mutant allele encoding a feedback-resistant aspartokinase would also exhibit synthetic lethality with a hom6Δ mutant.

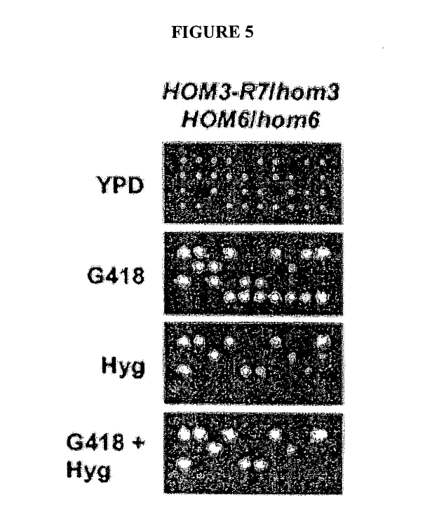

A yeast strain expressing a feedback resistant aspartokinase was constructed by integrating the HOM3-R7 mutant allele, which was originally isolated from a threonine-overproducing yeast strain as described in Pedersen et al., Mol. Gen. Genet. 1997 255, 561-569. The HOM3-R7 mutant strain was then crossed with a hom3 hom6 double mutant to obtain a HOM3-R7/hom3::Kan HOM6/hom6::hyg diploid strain. Tetrad analysis of this diploid revealed that most HOM3-R7 hom6Δ recombinants were inviable, and those few that did survive grew poorly. Taken together, these results indicate that dysregulation of aspartokinase activity becomes toxic to cells defective in homoserine dehydrogenase activity. (FIG. 5)

### EXAMPLE 6

FKBP12-aspartokinase interactions were studied in vitro by affinity chromatography by assaying binding of wild-type and mutant aspartokinase to recombinant His₆-Fpr1 protein that had been produced in bacteria and coupled to an aragose matrix. Both wild-type and feedback-resistant aspartokinase interacted with FKBP12 in the absence of exogenous threonine. Addition of threonine to the binding reactions increased FKBP12 interaction with wild-type aspartokinase but not with feedback resistant aspartokinase.

The two hybrid host strain PJ69-4A, coexpressing Ga14 DNA binding Domain-Fpr1(Ga14BD-Fpr1) and Ga14 activation domain-aspartokinase (Ga14AD-AK) fusion proteins, was grown in a synthetic medium with or without threonine. Interactions between the fusion proteins were detected and quantified by measuring expression of the lacZ reporter gene. Similar experiments were conducted with cells coexpressing Ga14BD-Fpr1 and a Ga14AD-AK^{E282D} feedback resistant fusion protein. In the absence of exogenous threonine, the interaction detected between FKBP12 and wild-type aspartokinase was considerably greater than that detected between AK^{E282D}, indicating that the mutant aspartokinase binds FKBP12 with less affinity than wild-type aspartokinase in vivo in the presence of threonine at normal intracellular concentrations. Addition, of threonine to the culture medium increased both FKBP12-AK and FKBP12-AK^{E282D} binding, lending support that threonine enhances these interactions. FKBP12-aspartokinase binding was nearly abolished by the presence of FK506.

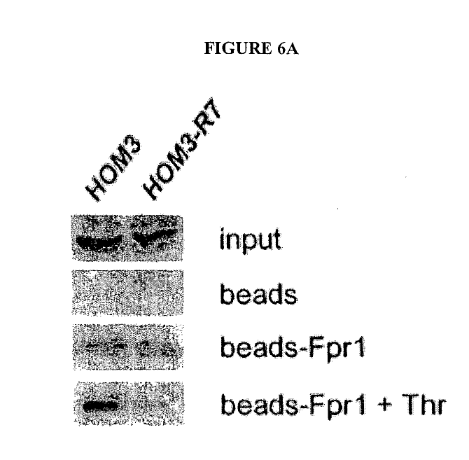

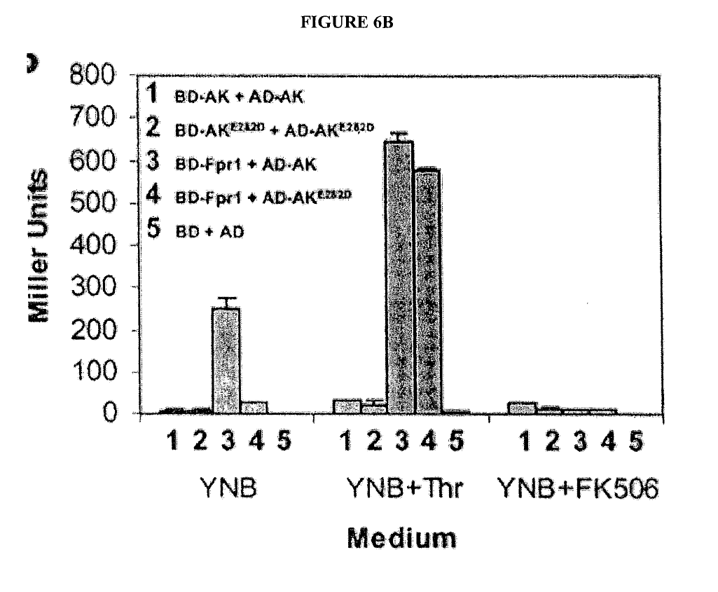

A low but significant level of self-interaction between both wild-type and mutant aspartokinases were detected in the two hybrid assay suggesting yeast aspartokinase forms dimmers of oligomers. These interactions were detected in the presence of FK506 indicating that neither FKBP12 bindings nor prolyl-isomerase activity is required for aspartokinase self-interaction. (FIGS. 6A and 6B)

### EXAMPLE 7

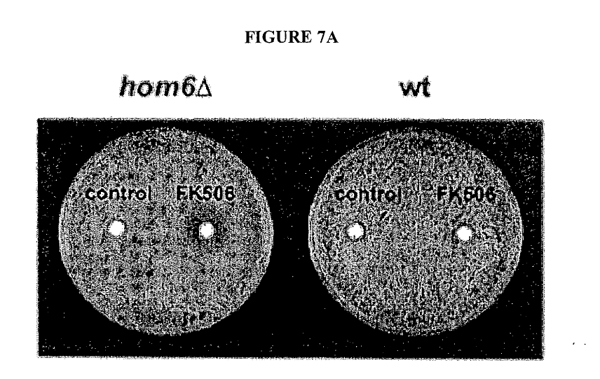

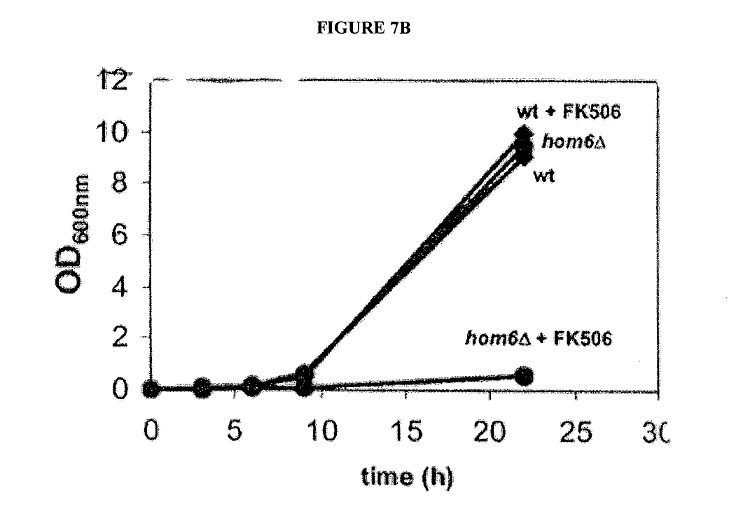

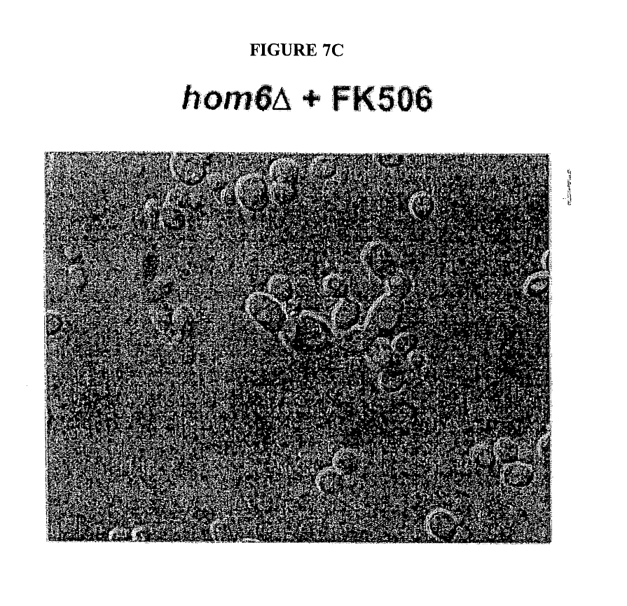

The hom6Δ mutation confers sensitivity to FK506. FK506 mimics the lethal effect of an fpr1Δ mutation in a homoserine dehydrogenase-deficient mutant. By microscopic examination, hom6Δ mutant cells exposed to FK506 exhibited abnormal morphologies, indicative of a cytokinesis defect. (FIGS. 7A, 7B, and 7C).

### EXAMPLE 8

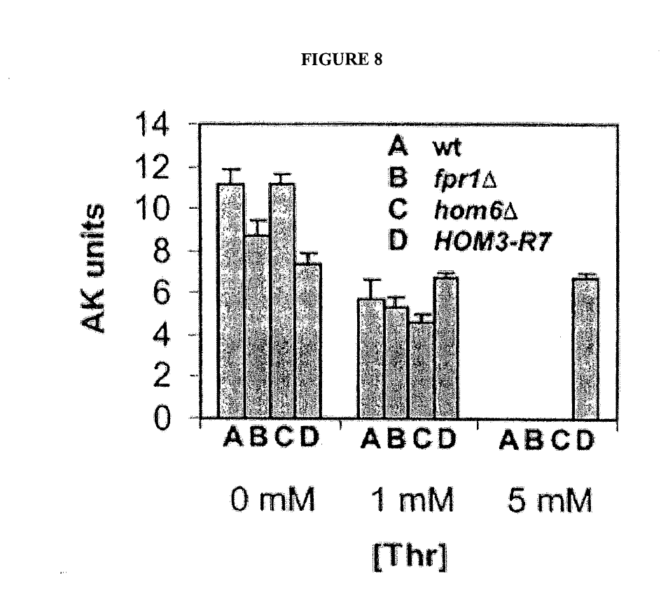

Aspartokinase from and fpr1 mutant is inhibited by threonine in vitro. Aspartokinase activity was assayed in the presence and absence of threonine from wild-type and fpr1, hom6 or HOM3-R7 feedback-resistant mutant cells. Aspartokinase expression and aspartokinase partial purification was analyzed by Western blotting, revealing no significant differences between the strains. All strains exhibited similar levels of aspartokinase activity in the absence of threonine. In the presence of 1 mM threonine, the aspartokinase activity from wild-type and fpr1Δ strains were inhibited to comparable levels, indicating that FKBP12 is not required for aspartokinase inhibition in vitro by threonine. Threonine also inhibited aspartokinase activity of the hom6Δ mutant suggesting that homoserine dehydrogenase does not play a role in aspartokinase feedback inhibition. Aspartokinase activity was completely feedback inhibited by the addition of 5 mM threonine, whereas the feedback-resistant aspartokinase exhibited no inhibition. Addition of recombinant His6-Frp1 to these assays did not have any detectable effect on feedback inhibition of aspartokinase form wild-type or the fpr1Δ mutant. (FIGS. 8A and 8B)

We assayed aspartokinase from the wild-type and fpr1Δ strains following preincubation of the enzyme preparations with threonine in the presence or absence of bacterially expressed His₆-Frp1. Threonine-treated aspartokinase preparations were mixed with the aspartokinase assay reagents, and the aspartokinase activity was compared to that of similar aspartokinase reactions containing the same final threonine concentrations but in which aspartokinase was not preexposed to threonine. No increase in threonine inhibition was revealed. In these assays, there was no significant difference in threonine inhibition of aspartokinase form the wild type compared to results with the fpr1 mutant.

What is claimed is:

1. A method for treating a pathogenic fungal infection in a mammalian subject comprising the step of administering to the subject a composition comprising a FKBP12 inhibitor and a homoserine dehydrogenase inhibitor.

2. The method of claim 1 wherein said FKBP12 inhibitor is non-immunosuppressive.

3. The method of claim 1 wherein said mammalian subject is a human patient.

4. The method of claim 3 wherein said human patient is immunocompromised.

5. The method of claim 4 wherein said human patient is immunocompromised as a result of drug or radiation treatment.

6. The method of claim 4 in which said human patient is a recipient, or is being prepared to become a recipient, of a tissue or organ transplant.

7. The method of claim 1 in which said composition comprising a FKBP12 inhibitor and a homoserine dehydrogenase inhibitor are administered in conjunction with an immunosuppressive agent.

8. The method of claim 4 wherein said human patient is immunocompromised as a result of diabetes, HIV infection, or other disease or disorder.

9. The method of claim 1 wherein said pathogenic fungal infection is caused by a fungus selected from the group consisting of: Candida species; Aspergillus species; Cryptococcus neofomans; Blastomyces species; Pneumocystis carinii; Coccidioides immitis; Basidiobolus ranarum; Conidiobolus species; Histoplasma capsulatum; Rhizopus species; Cunninghamella species; Rhizomucor species; Paracoccidioides brasiliensis; Pseudallescheria boydii; Rhinosporidium seeberi; or Sporothrix schenckii.

10. A method for treating a pathogenic fungal infection comprising providing a mammalian subject infected with a fungus resistant to one or more other antifungal therapies and administering to said subject a composition comprising a FKBP12 inhibitor and a homoserine dehydrogenase inhibitor.

11. The method of claim 10 wherein said fungus is resistant to one or more of amphotericin B, an analog thereof or another polyene macrolide antibiotic; flucytosine; griseofulvin; or an imidazole or triazole.

12. The method of claim 11 wherein said imidazole or triazole is clotrimazole, miconazole, ketoconazole, econazole, butoconazole, oxiconazole, terconazole, itraconazole or fluconazole.

13. The method of claim 10 wherein said composition comprising a FKBP12 inhibitor and a homoserine dehydrogenase inhibitor are administered to said subject in conjunction with at least one other antifungal agent.

14. A method of claim 13 in which said other antifungal agent is amphotericin B, an analog thereof or another polyene macrolide antibiotic; flucytosine; griseofulvin; or an imidazole or triazoles such as, e.g., clotrimazole, miconazole, ketoconazole, econazole, butoconazole, oxiconazole, terconazole, itraconazole or fluconazole.

15. A method for treating a pathogenic fungal infection in a mammalian subject comprising the step of administering to the subject a composition comprising:
a) a compound selected from FK506, L-683,590, L-685,818, and rapamycin; and
b) RI-331.
